(19) United States
(12) Patent Application Publication
(10) Pub. No.: US 2014/0093564 A1
(43) Pub. Date: Apr. 3, 2014

(54) PHARMACEUTICAL COMPOSITIONS OF COMBINATIONS OF DIPEPTIDYL PEPTIDASE-4 INHIBITORS WITH SIMVASTATIN

(71) Applicants: Kathryn Bradley, Hoddesdon (GB); Richard Elkes, Hoddesdon (GB); Shaun Fitzpatrick, Hoddesdon (GB); Robert Saklatvala, Rahway, NJ (US); Mustafa Mohamed, Slough (GB); Richard Kendall, Hoddesdon (GB)

(72) Inventors: Kathryn Bradley, Hoddesdon (GB); Richard Elkes, Hoddesdon (GB); Shaun Fitzpatrick, Hoddesdon (GB); Robert Saklatvala, Rahway, NJ (US); Mustafa Mohamed, Slough (GB); Richard Kendall, Hoddesdon (GB)

(73) Assignees: MERCK SHARP & DOHME LTD., Hoddesdon (GB); Merck Sharp & Dohme Corp, Rahway, NJ (US)

(21) Appl. No.: 14/118,071

(22) PCT Filed: Jun. 8, 2012
(86) PCT No.: PCT/US12/41446

§ 371 Date: Nov. 15, 2013

Related U.S. Application Data
(60) Provisional application No. 61/496,662, filed on Jun. 14, 2011.

(51) Int. Cl. A61K 9/24; A61K 31/366; A61K 31/4985

(52) U.S. Cl. CPC A61K 9/209; A61K 31/4985; A61K 31/366
USPC 424/465; 514/249

(57) ABSTRACT

The present invention is directed to novel pharmaceutical compositions comprising fixed dose combinations of a dipeptidyl peptidase-4 inhibitor (DPP-4 inhibitor), or a pharmaceutically acceptable salt thereof, and simvastatin, or pharmaceutically acceptable salt thereof, methods of preparing such pharmaceutical compositions, and methods of treating Type 2 diabetes and hypercholesterolemia with such pharmaceutical compositions. In particular, the invention is directed to pharmaceutical compositions comprising fixed-dose combinations of sitagliptin phosphate and simvastatin.

### BACKGROUND OF THE INVENTION

Type 2 diabetes is a chronic and progressive disease arising from a complex pathophysiology involving the dual endocrine defects of insulin resistance and impaired insulin secretion. The treatment of Type 2 diabetes typically begins with diet and exercise, followed by oral antidiabetic monotherapy. Many patients with type 2 diabetes are considered to be at high risk for coronary artery disease and associated co-morbidities. Coronary artery disease is a multifactorial disease in which the incidence and severity are affected by a myriad of factors such as lipid profile, presence of diabetes and the sex of the patient. In order to meaningfully reduce the risk of coronary artery disease, it is crucial to manage the entire risk spectrum. Treatment with cholesterol synthesis inhibitors in patients with and without coronary artery disease or coronary heart disease reduces the risk of cardiovascular morbidity and mortality.

For many patients at risk of a coronary heart disease event due to existing diabetes, treatment with both an anti-diabetic agent and a cholesterol synthesis inhibitor is recommended. However, co-prescription of an oral anti-diabetic drug and an oral cholesterol synthesis inhibitor may result in treatment regimens that are complex and difficult for many patients to follow. Combining an anti-diabetic agent and a cholesterol synthesis inhibitor into a single tablet provides a potential means of delivering combination therapy without adding to the complexity of patients' daily regimens. Such formulations have been well accepted in other disease indications, such as hypertension (HYZAAR™ which is a combination of losartan potassium and hydrochlorothiazide) and cholesterol lowering (VYTORIN™ which is a combination of simvastatin and ezetimibe). The selection of effective and well-tolerated treatments is a key step in the design of a combination tablet. Moreover, it is essential that the components have complementary mechanisms of action and compatible pharmacokinetic profiles. Examples of marketed combination tablets containing two oral antidiabetic agents include Glucovance™ (metformin and glyburide), Avandamet™ (metformin and rosiglitazone), and Metaglip™ (metformin and glipizide).

Currently sitagliptin phosphate monohydrate is available as a separate tablet for the treatment of type 2 diabetes. Simvastatin is also currently available as a separate tablet for the treatment of hypercholesterolemia. This invention provides a pharmaceutical composition comprising sitagliptin, or a pharmaceutically acceptable salt thereof, and simvastatin in a single bilayer tablet for superior efficacy, stability, patient convenience and compliance for the treatment of type 2 diabetes and hypercholesterolemia. This invention further provides a pharmaceutical composition in a single bilayer tablet comprising sitagliptin, or a pharmaceutically acceptable salt thereof, and simvastatin in which the sitagliptin and simvastatin chemical interactions and contact surface are minimized. This invention further provides a pharmaceutical composition in a single bilayer tablet comprising sitagliptin, or a pharmaceutically acceptable salt thereof, and simvastatin in which the adhesion of the sitagliptin layer and simvastatin layer is improved, resulting in a more stable bilayer tablet during compression, and downstream processing.

Simvastatin (ZOCOR®) is a HMG-CoA reductase inhibitor (statin) used to lower blood cholesterol by reducing the production of cholesterol by the liver. The conversion of 3-hydroxy-3-methylglutaryl-coenzyme A (HMG-CoA) to mevalonate is an early rate-limiting step in the cholesterol biosynthetic pathway. This step is catalyzed by the enzyme HMG-CoA reductase. Because statins inhibit HMG-CoA reductase from catalyzing this conversion, they act as potent lipid lowering agents. Statins are thus useful for the prevention and treatment of those conditions caused or exacerbated by high levels of cholesterol. In particular, in patients at high risk of coronary events because of existing coronary heart disease, diabetes and peripheral vessel disease, a history of stroke or other cerebrovascular disease, simvastatin is indicated to reduce the risk of total mortality by reducing coronary heart disease deaths, by reducing the risk of non-fatal myocardial infarction and stroke, and by reducing the need for coronary and non-coronary revascularization procedures in patients at high risk of coronary events.

Dipeptidyl peptidase-4 (DPP-4) inhibitors represent a novel class of agents that are being developed for the treatment or improvement in glycemic control in patients with Type 2 diabetes. Specific DPP-4 inhibitors currently in clinical trials for the treatment of Type 2 diabetes include sitagliptin phosphate (MK-0431), vildagliptin (LAF-237), saxagliptin (BMS-47718), alogliptin, carmegliptin, melogliptin, dutogliptin, denagliptin, linagliptin, P93/01 (Prosidion), SYR322 (Takeda), GSK 823093, Roche 0730699, TS021 (Taisho), E3024 (Eisai), and PHX-1149 (Phenomix). For example, oral administration of vildagliptin or sitagliptin to human Type 2 diabetics has been found to reduce fasting glucose and postprandial glucose excursion in association with significantly reduced HbA_{1c} levels. For reviews on the application of DPP-4 inhibitors for the treatment of Type 2 diabetes, reference is made to the following publications: (1) H.-U. Demuth, et al., “Type 2 diabetes—Therapy with dipeptidyl peptidase IV inhibitors, Biochim. Biophys. Acta, 1751: 33-44 (2005) and (2) K. Augustyns, et al., “Inhibitors of proline-specific dipeptidyl peptidases: DPP IV inhibitors as a novel approach for the treatment of Type 2 diabetes,” Expert Opin. Ther. Patents, 15: 1387-1407 (2005).

Sitagliptin phosphate having structural formula I below is the dihydrogen phosphate salt of (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine.

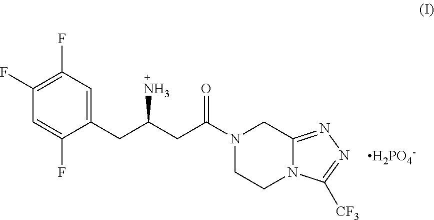

In one embodiment, sitagliptin phosphate is in the form of a crystalline anhydrate or monohydrate. In a class of this embodiment, sitagliptin phosphate is in the form of a crystalline monohydrate. Sitagliptin free base and pharmaceutically acceptable salts thereof are disclosed in U.S. Pat. No. 6,699,871, the contents of which are hereby incorporated by reference in their entirety. Crystalline sitagliptin phosphate monohydrate is disclosed in international patent publication WO 2005/0031335 published on Jan. 13, 2005. For a review on sitagliptin phosphate (MK-0431) including its synthesis and pharmacological properties, reference is made to the following publications: (1) C. F. Deacon, “MK-431,” Curr. Opin. Invest. Drugs, 6: 419-426 (2005) and (2) “MK-0431”, Drugs of the Future,” 30: 337-343 (2005).

Vildagliptin (LAF-237) is the generic name for (S)-1-[(3-hydroxy-1-adamantyl)amino]acetyl-2-cyano-pyrrolidine having structural formula II. Vildagliptin is specifically disclosed in U.S. Pat. No. 6,166,063, the contents of which are hereby incorporated by reference in their entirety.

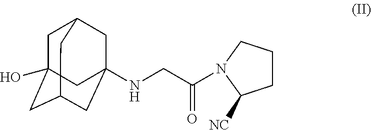 [SMILES: N#C[C@@H]1CCCN1C(=O)CNC12CC3CC(CC(O)(C3)C1)C2]

Saxagliptin (BMS-47718) is a methanoprolinenitrile of structural formula III below. Saxagliptin is specifically disclosed in U.S. Pat. No. 6,395,767, the contents of which are hereby incorporated by reference in their entirety.

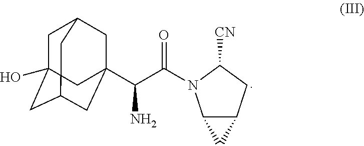 [SMILES: N#C[C@@H]1C[C@@H]2C[C@@H]2N1C(=O)[C@@H](N)C12CC3CC(CC(O)(C3)C1)C2]

Alogliptin (SYR-322) is a DP-IV inhibitor under investigation for the treatment of type 2 diabetes of structural formula IV below:

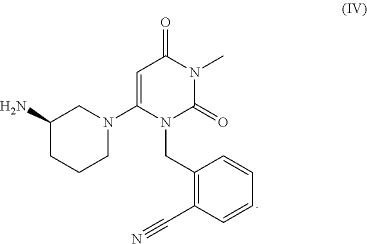 [SMILES: Cn1c(=O)cc(N2CCC[C@@H](N)C2)n(Cc2ccccc2C#N)c1=O]

Other DPP-4 inhibitors useful in the formulation of the present invention include, but are not limited to: alogliptin, carmegliptin, melogliptin, dutogliptin, denagliptin, linagliptin, saxagliptin and vildagliptin.

The present invention provides for pharmaceutical compositions of a fixed-dose combination of a dipeptidyl peptidase-4 inhibitor (DPP-4 inhibitor) and simvastatin which are prepared by dry and/or wet processing methods.

The present invention also provides a process to prepare pharmaceutical compositions of a fixed-dose combination of a DPP-4 inhibitor and simvastatin by dry and wet processing methods.

Another aspect of the present invention provides methods for the treatment of Type 2 diabetes and hypercholesterolemia by administering to a patient in need of such treatment a therapeutically effective amount of a pharmaceutical composition of the present invention.

These and other aspects will become readily apparent from the detailed description which follows.

### SUMMARY OF THE INVENTION

The present invention is directed to novel pharmaceutical compositions comprising fixed dose combinations of a dipeptidyl peptidase-4 inhibitor (DPP-4 inhibitor), or a pharmaceutically acceptable salt thereof, and simvastatin, or pharmaceutically acceptable salt thereof, methods of preparing such pharmaceutical compositions, and methods of treating Type 2 diabetes and hypercholesterolemia with such pharmaceutical compositions. In particular, the invention is directed to pharmaceutical compositions comprising fixed-dose combinations of sitagliptin phosphate and simvastatin.

### DETAILED DESCRIPTION OF THE INVENTION

One aspect of the present invention is directed to dosage forms for the medicinal administration of a fixed-dose combination of a dipeptidyl peptidase-4 inhibitor (DPP-4 inhibitor), or a pharmaceutically acceptable salt thereof, and simvastatin, or a pharmaceutically acceptable salt thereof. Such dosage forms may be in the powder or solid format including, but not limited to, tablets, capsules, and sachets. A particular solid dosage form relates to tablets comprising a fixed-dose combination of a DPP-4 inhibitor and simvastatin (also known as butanoic acid, 2,2-dimethyl-,1,2,3,7,8,8a-hexahydro-3,7-dimethyl-8-[2-(tetrahydro-4-hydroxy-6-oxo-2H-pyran-2-yl)-ethyl]-1-naphthalenyl ester, [1S-[1α,3α,7β,8β(2S*,4S*),-8aβ]]):

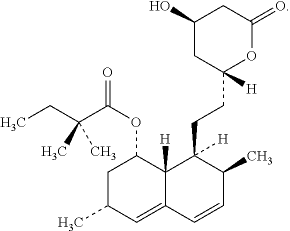

Simvastatin is marketed as ZOCOR® in lactone pro-drug form and functions as a HMG-CoA reductase inhibitor after administration. Pharmaceutically acceptable salts of simvastatin include, but are not limited to, the pharmaceutically acceptable salts of dihydroxy open ring acid.

In one embodiment of the present invention, the simvastatin active pharmaceutical ingredient (API) includes butylated hydroxyanisole (BHA). In a class of this embodiment, the simvastatin API includes less than 1% of butylated hydroxy anisole. In another class of this embodiment, the simvastatin API includes less than 0.05% of butylated hydroxyanisole. In another class of this embodiment, the simvastatin API includes about 0.01% of butylated hydroxy anisole.

In a particular aspect of the present invention, the pharmaceutical compositions in the form of a bilayer tablet comprise: (a) a first layer comprising a dipeptidyl peptidase-4 inhibitor, or a pharmaceutically acceptable salt thereof; and (b) a second layer comprising simvastatin. In one embodiment of the present invention, the first bilayer additionally comprises one or more excipients selected from the group consisting of (i) a diluent; (ii) a disintegrant; and (iii) a lubricant. In another embodiment of the present invention, the second bilayer additionally comprises one or more excipients selected from the group consisting of: (i) a diluent, (ii) an anti-oxidant; (iii) a binding agent; and (iv) a lubricant. In another embodiment of the present invention, the pharmaceutical compositions may also contain one or more surfactants or wetting agents; and one or more antioxidants.

In another embodiment of this aspect of the invention, the DPP-4 inhibitor is selected from the group consisting of sitagliptin, vildagliptin, saxagliptin, P93/01, SYR322, GSK 823093, Roche 0730699, TS021, E3024, and PHX-1149. In a class of this embodiment the DPP-4 inhibitor is alogliptin, carmegliptin, melogliptin, dutogliptin, denagliptin, linagliptin, sitagliptin, vildagliptin, or saxagliptin. In a subclass of this class, the DPP-4 inhibitor is sitagliptin.

A preferred pharmaceutically acceptable salt of sitagliptin is the dihydrogen phosphate salt of structural formula I above (sitagliptin phosphate). A preferred form of the sitagliptin dihydrogen phosphate salt is the crystalline monohydrate (sitagliptin phosphate monohydrate) disclosed in WO 2005/0031335.

The preparation of sitagliptin and pharmaceutically acceptable salts thereof is disclosed in U.S. Pat. No. 6,699,871, the contents of which are herein incorporated by reference in their entirety. The preparation of sitagliptin phosphate monohydrate is disclosed in international patent publication WO 2005/0031335 published on Jan. 13, 2005, the contents of which are herein incorporated by reference in their entirety.

The dosage strength of the DPP-4 inhibitor for incorporation into the pharmaceutical compositions of the present invention is an amount from about 1 milligram to about 250 milligrams of the active moiety. A preferred dosage strength of the DPP-4 inhibitor is an amount from about 25 milligrams to about 200 milligrams of the active moiety. Discrete dosage strengths are the equivalent of 25, 50, 75, 100, 150, and 200 milligrams of the DPP-4 inhibitor active moiety. By “active moiety” is meant the free base form of the DPP-4 inhibitor as an anhydrate.

The unit dosage strength of sitagliptin free base anhydrate (active moiety) for inclusion into the fixed-dose combination pharmaceutical compositions of the present invention is 25, 50, 75, 100, 150, or 200 milligrams. An equivalent amount of sitagliptin phosphate monohydrate to the sitagliptin free base anhydrate is used in the pharmaceutical compositions, namely, 32.13, 64.25, 96.38, 128.5, 192.75, and 257 milligrams, respectively. A preferred dosage strength of sitagliptin is 50 or 100 milligrams. Another preferred dosage strength of sitagliptin is 100 milligrams. Another preferred dosage strength of sitagliptin is 50 milligrams.

The dosage strength of simvastatin for incorporation into the pharmaceutical compositions of the present invention is an amount from about 1 milligram to about 100 milligrams of the active moiety. A preferred dosage strength of simvastatin is an amount from about 5 milligrams to about 80 milligrams of the active moiety. Discrete dosage strengths are the equivalent of 5, 10, 20, 40, and 80 milligrams of simvastatin. Another preferred dosage strength of simvastatin is 5, 10, 20, 40 or 80 milligrams of simvastatin. Another preferred dosage strength of simvastatin is 10, 20, 40 or 80 milligrams of simvastatin. Another preferred dosage strength of simvastatin is 10, 20, or 40 milligrams of simvastatin.

The unit dosage strength of simvastatin for inclusion into the fixed-dose combination pharmaceutical compositions of the present invention is 5 milligrams, 10 milligrams, 20 milligrams, 40 milligrams and 80 milligrams. In one embodiment, the simvastatin in the unit dosage strengths 5 mg, 10 mg, 20 mg, 40 mg and 80 mg may contain between 0.001% and 0.1% butylated hydroxyanisole. In a class of this embodiment, the simvastatin in the unit dosage strengths 5 mg, 10 mg, 20 mg, 40 mg and 80 mg may contain about 0.01% of butylated hydroxyanisole. These unit dosage strengths of simvastatin represent the dosage strengths approved in the U.S. for marketing to treat Type 2 diabetes.

Specific embodiments of dosage strengths for sitagliptin and simvastatin in the fixed-dose combinations of the present invention are the following:
(1) 100 milligrams of sitagliptin (equivalent to 128.5 milligrams of sitagliptin phosphate monohydrate) and 5 milligrams simvastatin;
(2) 100 milligrams of sitagliptin (equivalent to 128.5 milligrams of sitagliptin phosphate monohydrate) and 10 milligrams simvastatin;
(3) 100 milligrams of sitagliptin (equivalent to 128.5 milligrams of sitagliptin phosphate monohydrate) and 20 milligrams simvastatin; and
(4) 100 milligrams of sitagliptin (equivalent to 128.5 milligrams of sitagliptin phosphate monohydrate) and 40 milligrams simvastatin;
(5) 100 milligrams of sitagliptin (equivalent to 128.5 milligrams of sitagliptin phosphate monohydrate) and 80 milligrams simvastatin;
(6) 50 milligrams of sitagliptin (equivalent to 64.26 milligrams of sitagliptin phosphate monohydrate) and 5 milligrams simvastatin;
(7) 50 milligrams of sitagliptin (equivalent to 64.26 milligrams of sitagliptin phosphate monohydrate) and 10 milligrams simvastatin;
(8) 50 milligrams of sitagliptin (equivalent to 64.26 milligrams of sitagliptin phosphate monohydrate) and 20 milligrams simvastatin;
(9) 50 milligrams of sitagliptin (equivalent to 64.26 milligrams of sitagliptin phosphate monohydrate) and 40 milligrams simvastatin, and
(10) 50 milligrams of sitagliptin (equivalent to 64.26 milligrams of sitagliptin phosphate monohydrate) and 80 milligrams simvastatin.

The pharmaceutical compositions of the present invention are prepared by dry/or and wet processing methods. In one embodiment the simvastatin layer is prepared by wet processing methods. In a class of this embodiment, the simvastatin layer is prepared by wet granulation methods. With wet granulation either high-shear granulation or fluid-bed granulation may be used. In another class of this embodiment, the simvastatin layer is prepared by high-shear wet granulation.

In another embodiment the DPP-4 layer is prepared by dry processing methods. In a class of this embodiment, the DPP-4 layer is prepared by direct compression or direct compression blend. In another class of this embodiment, the DPP-4 layer is prepared by direct dry compression.

The pharmaceutical compositions obtained by dry and wet processing methods may be compressed into tablets using a bilayer press, encapsulated, or metered into sachets, and optionally film coated.

The pharmaceutical compositions contain one or more lubricants or glidants. Examples of lubricants include magnesium stearate, magnesium stearate (non-bovine), calcium stearate, stearic acid, sodium stearyl fumarate, hydrogenated castor oil, and mixtures thereof. In one embodiment, the lubricant is magnesium stearate or sodium stearyl fumarate, or a mixture thereof. In another embodiment, the lubricant is a mixture of magnesium stearate and sodium stearyl fumarate. In another embodiment, the lubricant is magnesium stearate. In another embodiment, the lubricant is sodium stearyl fumarate. Examples of glidants include colloidal silicon dioxide, calcium phosphate tribasic, magnesium silicate, and talc.

The pharmaceutical compositions of the present invention optionally contain one or more binding agents. Embodiments of binding agents include hydroxypropylcellulose (HPC), hydroxypropylmethyl cellulose (HPMC), hydroxyethyl cellulose, pregelatinized starch, pregelatinized corn starch, starch 1500, corn starch, polyvinylpyrrolidone (povidone), and co-povidone. In one embodiment, the binding agent is polyvinylpyrrolidone. In another embodiment, the binding agent is hydroxypropylcellulose (HPC). In another embodiment, the binding agent is hydroxypropylcellulose (HPC) in solution. In another embodiment, the binding agent is hydroxypropylcellulose (HPC) in an aqueous solution. In another embodiment, the binding agent is hydroxypropylcellulose (HPC). In another embodiment, the binding agent is pregelatinized starch or pregelatinized corn starch, or a mixture thereof. In another embodiment, the binding agent is pregelatinized starch. In another embodiment, the binding agent is pregelatinized corn starch. In another embodiment, the pregelatinized starch is starch 1500. In another embodiment, the pregelatinized corn starch is starch 1500.

The pharmaceutical compositions of the present invention may also optionally contain one or more diluents. Examples of diluents include mannitol, sorbitol, anhydrous dibasic calcium phosphate, lactose monohydrate, dibasic calcium phosphate dihydrate, microcrystalline cellulose, and powdered cellulose. In one embodiment the diluent is selected from: mannitol, anhydrous dibasic calcium phosphate, lactose monohydrate and microcrystalline cellulose, or a mixture of any two, three or four thereof. In one embodiment the diluent is selected from: anhydrous dibasic calcium phosphate, lactose monohydrate and microcrystalline cellulose, or a mixture of any two or three thereof. In another embodiment the diluent is selected from: anhydrous dibasic calcium phosphate and microcrystalline cellulose, or a mixture thereof. In another embodiment the diluent is a mixture of anhydrous dibasic calcium phosphate and microcrystalline cellulose.

In another embodiment of the present invention, the diluent in the first layer is a mixture with a ratio of about 0:4 to about 2:6 of microcrystalline cellulose to anhydrous dibasic calcium phosphate. In another embodiment of the present invention, the diluent in the first layer is a mixture with a ratio of about 0.5:4.5 to about 1.5:5.5 of microcrystalline cellulose to anhydrous dibasic calcium phosphate. In another embodiment of the present invention, the diluent in the first layer is a mixture with a ratio of about 1:4 to about 1:6 of microcrystalline cellulose to anhydrous dibasic calcium phosphate. In another embodiment of the present invention, the diluent in the first layer is a mixture with a ratio of about 1 to about 5 of microcrystalline cellulose to anhydrous dibasic calcium phosphate. In another embodiment of the present invention, the diluent in the first layer is a mixture of 1:5.19 microcrystalline cellulose to anhydrous dibasic calcium phosphate.

In another embodiment of the present invention, the diluent in the first layer is a mixture with a ratio of about 0:4 to about 2:6 of microcrystalline cellulose to anhydrous dibasic calcium phosphate; the disintegrant is croscarmellose sodium; and the lubricant is a mixture of magnesium stearate and sodium stearyl fumarate. In another embodiment of the present invention, the diluent in the first layer is a mixture with a ratio of about 0.5:4.5 to about 1.5:5.5 of microcrystalline cellulose to anhydrous dibasic calcium phosphate; the disintegrant is croscarmellose sodium; and the lubricant is a mixture of magnesium stearate and sodium stearyl fumarate. In another embodiment of the present invention, the diluent in the first layer is a mixture with a ratio of about 1:4 to about 1:6 of microcrystalline cellulose to anhydrous dibasic calcium phosphate; the disintegrant is croscarmellose sodium; and the lubricant is a mixture of magnesium stearate and sodium stearyl fumarate. In another embodiment of the present invention, the diluent in the first layer is a mixture with a ratio of about 1 to about 5 of microcrystalline cellulose to anhydrous dibasic calcium phosphate; the disintegrant is croscarmellose sodium; and the lubricant is a mixture of magnesium stearate and sodium stearyl fumarate. In another embodiment of the present invention, the diluent in the first layer is a mixture of 1:5.19 microcrystalline cellulose to anhydrous dibasic calcium phosphate; the disintegrant is croscarmellose sodium; and the lubricant is a mixture of magnesium stearate and sodium stearyl fumarate.

It was unexpectedly found that decreasing the amount of microcrystalline cellulose relative to the amount of dibasic calcium phosphate in the diluent portion of the sitagliptin layer reduced the unequal swelling of the two layers of the bilayer tablet and reduced the resulting bilayer delamination. To improve the adhesion of the layers in the bilayer tablet and to decrease the swelling of the sitagliptin layer relative to the simvastatin layer in the bilayer tablet, the amount of dibasic calcium phosphate is greater than the amount of microcrystalline cellulose. Preferably the amount of dibasic calcium phosphate is about five times the amount of microcrystalline cellulose in the diluent portion of the sitagliptin layer of the bilayer tablet.

In another embodiment, the diluent is selected from: lactose monohydrate and microcrystalline cellulose, or a mixture thereof. In another embodiment the diluent is a mixture of lactose monohydrate and microcrystalline cellulose. In another embodiment, the diluent is microcrystalline cellulose. Microcrystalline cellulose is available from several suppliers and includes Avicel, Avicel PH 101, Avicel PH 102, Avicel, PH 103, Avicel PH 105, and Avicel PH 200, manufactured by the FMC Corporation. In another embodiment, the diluent is mannitol. In another embodiment, the diluent is a mixture of microcrystalline cellulose and mannitol. In another embodiment, the diluent is a 2:1 to 1:2 mixture of microcrystalline cellulose to mannitol. In another embodiment, the diluent is microcrystalline cellulose, mannitol and anhydrous dibasic calcium phosphate. In another embodiment, the diluent is microcrystalline cellulose or mannitol or anhydrous dibasic calcium phosphate. In another embodiment, the diluent is mannitol and anhydrous dibasic calcium phosphate. In another embodiment, the diluent is anhydrous dibasic calcium phosphate. In another embodiment of the present invention, the diluent is lactose monohydrate.

The pharmaceutical compositions of the present invention may also optionally contain a disintegrant. The disintegrant may be one of several modified starches, modified cellulose polymers, or polycarboxylic acids, such as croscarmellose sodium, sodium starch glycolate, polacrillin potassium, carboxymethylcellulose calcium (CMC Calcium), and crospovidone. In one embodiment, the disintegrant is selected from: polacrillin potassium, carboxymethylcellulose calcium (CMC Calcium), and crospovidone. In another embodiment, the disintegrant is crospovidone and croscarmellose sodium. In another embodiment, the disintegrant is crospovidone. In another embodiment, the disintegrant is croscarmellose sodium.

The pharmaceutical compositions of the present invention may also optionally contain one or more surfactants or wetting agents. The surfactant may be anionic, cationic, or neutral. Anionic surfactants include sodium lauryl sulfate, sodium dodecanesulfonate, sodium oleyl sulfate, and sodium laurate mixed with stearates and talc. Cationic surfactants include benzalkonium chlorides and alkyltrimethylammonium bromides. Neutral surfactants include glyceryl monooleate, polyoxyethylene sorbitan fatty acid esters, polyvinyl alcohol, and sorbitan esters. Embodiments of wetting agents include poloxamer, polyoxyethylene alkyl ethers, polyoxyethylene castor oil derivatives, and polyoxyethylene stearates.

The pharmaceutical compositions of the present invention may also optionally contain an anti-oxidant which may be added to the formulation to impart chemical stability. The anti-oxidant is selected from the group consisting of α-tocopherol, λ-tocopherol, δ-tocopherol, extracts of natural origin rich in tocopherol, citric acid (including anhydrous citric acid or hydrates thereof), citric acid monohydrate, ascorbic acid, L-ascorbic acid and its sodium or calcium salts, ascorbyl palmitate, propyl gallate, octyl gallate, dodecyl gallate, butylated hydroxytoluene (BHT), and butylated hydroxyanisole (BHA). In one embodiment, the antioxidant is butylated hydroxyanisole. In another embodiment, the antioxidant is citric acid. In another embodiment, the antioxidant is citric acid monohydrate. In another embodiment, the antioxidant is ascorbic acid. In another embodiment of the present invention, the anti-oxidant is a mixture of butylated hydroxyanisole, citric acid or citric acid monohydrate and ascorbic acid. In another embodiment of the present invention, the anti-oxidant is a mixture of butylated hydroxyanisole, citric acid and ascorbic acid. In another embodiment of the present invention, the anti-oxidant is a mixture of butylated hydroxyanisole, citric acid monohydrate and ascorbic acid.

Preferred dosage forms for the pharmaceutical compositions of the present invention are tablets which are prepared by compression methods. Such tablets may be film-coated such as with a mixture of hydroxypropylcellulose and hydroxypropylmethylcellulose containing titanium dioxide and/or other coloring agents, such as iron oxides, dyes, and lakes; a mixture of polyvinyl alcohol (PVA) and polyethylene glycol (PEG) containing titanium dioxide and/or other coloring agents, such as iron oxides, dyes, and lakes; or any other suitable immediate-release film-coating agent(s). The coat provides taste masking and additional stability to the final tablet. A commercial film-coating agent is Opadry® which is a formulated powder blend provided by Colorcon. Embodiments of Opadry® useful in the present invention include, but are not limited to, Opadry® I (HPC/HPMC), Opadry® 20A18334, Opadry® II, Opadry® II HP (PVA-PEG), Purple Opadry® II [85F170000], Beige Opadry® [85F170001], Opadry® Red, Opadry® Red-Orange, Opadry® II Red, Opadry® II Orange Beige, or another suitable Opadry® suspension (such as polyvinyl alcohol, polyethylene glycol, titanium dioxide, and talc, with or without colorants).

Finally, a sweetening agent and/or flavoring agent may be added if desired.

In one embodiment of the present invention, the pharmaceutical composition comprises:

(a) a first layer comprising about 20 to 45% by weight of a dipeptidyl peptidase-4 inhibitor, or a pharmaceutically acceptable salt thereof; and
(b) a second layer comprising about 5 to 15% by weight of simvastatin, or a pharmaceutically acceptable salt thereof.

In a class of this embodiment, the first layer additionally comprises one or more excipients selected from the group consisting of: (i) a diluent; (ii) a disintegrant; and (iii) a lubricant. In a subclass of this class, the first layer additionally comprises one or more excipients selected from the group consisting of: (i) two diluents; (ii) a disintegrant; and (iii) two lubricants.

In another class of this embodiment, the first layer additionally comprises one or more excipients selected from the group consisting of: (i) about 40-80% by weight of a diluent; (ii) about 0.5-6% by weight of a disintegrant; and (iii) about 0.75-10% by weight of a lubricant. In a subclass of this class, the first layer additionally comprises one or more excipients selected from the group consisting of: (i) about 40-80% by weight of two diluents; (ii) about 0.5-6% by weight of a disintegrant; and (iii) about 0.75-10% by weight of two lubricants.

In another class of this embodiment, the first layer additionally comprises one or more excipients selected from the group consisting of: (i) about 35-60% by weight of a first diluent; (ii) about 5-20% of a second diluent; (iii) about 0.5-6% by weight of a disintegrant; (iv) about 0.5-4% by weight of a first lubricant and (v) about 0.25-6% by weight of a second lubricant. In a subclass of this class, the first diluent is anhydrous dibasic calcium phosphate; the second diluent is microcrystalline cellulose; the disintegrant is croscarmellose sodium; the first lubricant is sodium stearyl fumarate; and the second lubricant is magnesium stearate.

In another class of this embodiment, the second layer additionally comprises one or more excipients selected from the group consisting of: (i) a diluent, (ii) an anti-oxidant; (iii) a binding agent; and (iv) a lubricant. In another class of this embodiment, the second layer additionally comprises one or more excipients selected from the group consisting of: (i) two diluents, (ii) three anti-oxidants; (iii) a binding agent; and (iv) a lubricant.

In another class of this embodiment, the second layer additionally comprises one or more excipients selected from the group consisting of: (i) about 65-85% by weight of a diluent; (ii) about 1-10% by weight of an anti-oxidant; (iii) about 5-15% by weight of a binding agent; and (iv) about 0.1-1.5% by weight of a lubricant. In a subclass of this class, the first diluent is lactose monohydrate; the second diluent is microcrystalline cellulose; the first anti-oxidant is butylated hydroxyanisole; the second anti-oxidant is ascorbic acid; the third anti-oxidant is citric acid or citric acid monohydrate; and the lubricant is magnesium stearate.

In another class of this embodiment, the dipeptidyl peptidase-4 inhibitor is selected from the group consisting of: alogliptin, carmegliptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogen phosphate salt thereof.

In a second embodiment of the present invention, the pharmaceutical composition comprises:

(a) a first layer comprising:

(i) about 20 to 45% by weight of a dipeptidyl peptidase-4 inhibitor, or a pharmaceutically acceptable salt thereof;

(ii) about 40 to 80% by weight of a diluent;

(iii) about 0.5 to 6% by weight of a disintegrant; and

(iv) about 0.75 to 10% by weight of a lubricant; and

(b) a second layer comprising:

(i) about 5 to 15% by weight of simvastatin, or a pharmaceutically acceptable salt thereof;

(ii) about 70 to 80% by weight of a diluent;

(iii) about 2 to 5% by weight of an anti-oxidant;

(iv) about 5 to 15% by weight of a binding agent, and

(v) about 0.1 to 1.5% by weight of a lubricant.

In another class of this embodiment, the first layer additionally comprises one or more excipients selected from the group consisting of: (i) about 40-80% by weight of two diluents; (ii) about 0.5-6% by weight of a disintegrant; and (iii) about 0.75-10% by weight of two lubricants.

In another class of this embodiment, the first layer additionally comprises one or more excipients selected from the group consisting of: (i) about 35-60% by weight of a first diluent; (ii) about 5-20% of a second diluent; (iii) about 0.5-6% by weight of a disintegrant; (iv) about 0.5-4% by weight of a first lubricant and (v) about 0.25-6% by weight of a second lubricant. In a subclass of this class, the first diluent is anhydrous dibasic calcium phosphate; the second diluent is microcrystalline cellulose; the disintegrant is croscarmellose sodium; the first lubricant is sodium stearyl fumarate; and the second lubricant is magnesium stearate.

In another class of this embodiment, the pharmaceutical composition comprises a first layer wherein the diluent is selected from the group consisting of: microcrystalline cellulose, mannitol and anhydrous dibasic calcium phosphate, or a mixture thereof; the disintegrant is selected from the group consisting of: crospovidone and croscarmellose sodium, or a mixture thereof; and the lubricant is selected from the group consisting of: magnesium stearate and sodium stearyl fumarate, or a mixture thereof. In another class of this embodiment, the pharmaceutical composition comprises a first layer wherein the diluent is a mixture of microcrystalline cellulose and anhydrous dibasic calcium phosphate; the disintegrant is croscarmellose sodium; and the lubricant is a mixture of magnesium stearate and sodium stearyl fumarate. In another class of this embodiment, the diluent in the first layer is a mixture with a ratio of about 1:5 to about 1:6 of microcrystalline cellulose to anhydrous dibasic calcium phosphate; the disintegrant is croscarmellose sodium; and the lubricant is a mixture of magnesium stearate and sodium stearyl fumarate. In another class of this embodiment, the pharmaceutical composition comprises a first layer wherein the diluent is a mixture of 1:5.19 microcrystalline cellulose to anhydrous dibasic calcium phosphate; the disintegrant is croscarmellose sodium; and the lubricant is a mixture of magnesium stearate and sodium stearyl fumarate.

In another class of this embodiment, the second layer additionally comprises one or more excipients selected from the group consisting of: (i) about 70-80% by weight of two diluents; (ii) about 2-5% by weight of three anti-oxidants; (iii) about 5-15% by weight of a binding agent, and (iv) about 0.1-1.5% by weight of a lubricant.

In another class of this embodiment, the pharmaceutical composition comprises a second layer wherein the diluent is selected from the group consisting of anhydrous dibasic calcium phosphate, lactose monohydrate, microcrystalline cellulose and mannitol, or a mixture thereof; the anti-oxidant is selected from the group consisting of: butylated hydroxyanisole, citric acid, citric acid monohydrate, ascorbic acid or a mixture thereof; the binding agent is pregelatinized corn starch or pregelatinized starch; and the lubricant is selected from the group consisting of: magnesium stearate and sodium stearyl fumarate, or a mixture thereof. In another class of this embodiment, the diluent selected from the group consisting of: lactose monohydrate and microcrystalline cellulose, or a mixture thereof; the anti-oxidant is selected from the group consisting of butylated hydroxyanisole, citric acid, citric acid monohydrate, ascorbic acid or a mixture thereof; the binding agent is pregelatinized corn starch or pregelatinized starch; and the lubricant is selected from the group consisting of: magnesium stearate and sodium stearyl fumarate, or a mixture thereof. In another subclass of this class, the pharmaceutical composition comprises a second layer wherein the diluent is a mixture of lactose monohydrate and microcrystalline cellulose; the anti-oxidant is a mixture of butylated hydroxyanisole, citric acid monohydrate and ascorbic acid; the binding agent is pregelatinized corn starch or pregelatinized starch; and the lubricant is a mixture of: magnesium stearate and sodium stearyl fumarate. In another subclass of this class, the pharmaceutical composition comprises a second layer wherein the diluent is a mixture of lactose monohydrate and microcrystalline cellulose; the anti-oxidant is a mixture of butylated hydroxyanisole, citric acid and ascorbic acid; the binding agent is pregelatinized corn starch or pregelatinized starch; and the lubricant is a mixture of: magnesium stearate and sodium stearyl fumarate.

In another class of this embodiment, the dipeptidyl peptidase-4 inhibitor is selected from the group consisting of: alogliptin, carmegliptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogen phosphate salt thereof.

In a third embodiment of the present invention, the pharmaceutical composition comprises:

(a) a first layer comprising:

(i) about 25 to 35% by weight of a dipeptidyl peptidase-4 inhibitor, or a pharmaceutically acceptable salt thereof;

(ii) about 50-70% by weight of a diluent;

(iii) about 1-4% by weight of a disintegrant; and

(iv) about 1.5-7% by weight of a lubricant; and

(b) a second layer comprising:

(i) about 9 to 11% by weight of simvastatin, or a pharmaceutically acceptable salt thereof;

(ii) about 73 to 77% by weight of a diluent;

(iii) about 2 to 5% by weight of an anti-oxidant;

(iv) about 9 to 11% by weight of a binding agent; and

(v) about 0.1 to 1.5% by weight of a lubricant.

In a class of this embodiment, the pharmaceutical composition comprises a first layer comprising: (i) about 25 to 35% by weight of a dipeptidyl peptidase-4 inhibitor, or a pharmaceutically acceptable salt thereof; (ii) about 50-70% by weight of two diluents; (iii) about 1-4% by weight of a disintegrant; and (iv) about 1.5-7% by weight of two lubricants.

In another class of this embodiment, the first layer additionally comprises one or more excipients selected from the group consisting of: (i) about 40-70% by weight of a first diluent; (ii) about 5-15% of a second diluent; (iii) about 1-4% by weight of a disintegrant; (iv) about 1-4% by weight of a first lubricant and (v) about 0.5-3% by weight of a second lubricant. In a subclass of this class, the first diluent is anhydrous dibasic calcium phosphate; the second diluent is microcrystalline cellulose; the disintegrant is croscarmellose sodium; the first lubricant is sodium stearyl fumarate; and the second lubricant is magnesium stearate.

In another class of this embodiment, the pharmaceutical composition comprises a first layer wherein the dipeptidyl peptidase-4 inhibitor is sitagliptin, or a pharmaceutically acceptable salt thereof; the diluent is a mixture of microcrystalline cellulose and anhydrous dibasic calcium phosphate; the disintegrant is croscarmellose sodium; and the lubricant is a mixture of magnesium stearate and sodium stearyl fumarate.

In a class of this embodiment, the pharmaceutical composition comprises a second layer comprising: (i) about 9 to 11% by weight of a simvastatin; (ii) about 73-77% by weight of two diluents; (iii) about 2-5% by weight of three anti-oxidants; (iv) about 9-11% by weight of a binding agent, and (v) about 0.1-1.5% by weight of a lubricant.

In another class of this embodiment, the pharmaceutical composition comprises a second layer comprising: (i) about 9 to 11% by weight of a simvastatin; (ii) about 70-78% by weight of the first diluent; (iii) about 1-10% by weight of the second diluent; (iv) about 0.1-0.5% by weight of the first anti-oxidant; (v) about 1-4% by weight of the second anti-oxidant; (vi) 0.5-2.5% by weight of the third anti-oxidant; (vii) about 9-11% by weight of a binding agent, and (viii) about 0.1-1.5% by weight of a lubricant. In a subclass of this class, the first anti-oxidant is butylated hydroxyanisole; the second anti-oxidant is ascorbic acid; the third anti-oxidant is citric acid or citric acid monohydrate; the first diluent is lactose monohydrate; the second diluent is microcrystalline cellulose; the lubricant is magnesium stearate; and the binding agent is pregelatinized starch or pregelatinized corn starch.

In another class of this embodiment, the pharmaceutical composition comprises a second layer wherein the diluent is a mixture of lactose monohydrate and microcrystalline cellulose; the anti-oxidant is a mixture of butylated hydroxyanisole, ascorbic acid, and citric acid or citric acid monohydrate; the binding agent is pregelatinized corn starch or pregelatinized starch; and the lubricant is magnesium stearate.

In another class of this embodiment, the dipeptidyl peptidase-4 inhibitor is selected from the group consisting of: alogliptin, carmegliptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogen phosphate salt thereof.

In another class of the embodiments of the present invention, the pharmaceutical composition contains about 20 to 45% by weight of sitagliptin dihydrogen phosphate. In a subclass of this class, the composition contains about 25 to 35% of sitagliptin dihydrogen phosphate. In another subclass of this class, the composition contains about 32 to 33% of sitagliptin dihydrogen phosphate. In another subclass of this class, the composition contains about 32.12% of sitagliptin dihydrogen phosphate.

In another class of the embodiments of the present invention, the pharmaceutical composition contains about 25 to 45% by weight of sitagliptin, or a pharmaceutically acceptable salt thereof. In a subclass of this class, the composition contains about 25 to 35% of sitagliptin, or a pharmaceutically acceptable salt thereof. In another subclass of this class, the composition contains about 32 to 33% of sitagliptin, or a pharmaceutically acceptable salt thereof. In another subclass of this class, the composition contains about 32.11% of sitagliptin, or a pharmaceutically acceptable salt thereof. In another subclass of this class, the composition contains about 32.12% of sitagliptin, or a pharmaceutically acceptable salt thereof. In another subclass of this class, the composition contains about 32.13% of sitagliptin, or a pharmaceutically acceptable salt thereof.

In another class of the embodiments of the present invention, the pharmaceutical composition contains about 5 to 15% by weight of simvastatin. In a subclass of this class, the composition contains about 8 to 12% of simvastatin. In another subclass of this class, the composition contains about 9 to 11% of simvastatin. In another subclass of this class, the composition contains about 10% of simvastatin.

In another class of the embodiments of the present invention, the first layer of the pharmaceutical composition (the sitagliptin layer) contains about 40 to 80% by weight of a diluent. In a subclass of this class, the composition contains about 50 to 70% of a diluent. In another subclass of this class, the composition contains about 61.87% of a diluent. In another subclass of this class, the composition contains about 61.88% of a diluent. In another subclass of this class, the composition contains about 20 to 60% of a first diluent; and contains about 5 to 20% of a second diluent. In another subclass of this class, the composition contains about 45 to 55% of a first diluent; and contains about 5 to 15% of a second diluent. In another subclass of this class, the composition contains about 51.88% of a first diluent. In another subclass of this class, the composition contains about 10% of a second diluent. In another subclass of this class, the composition contains about 51.87% of a first diluent. In another subclass of this class, the diluent is microcrystalline cellulose or anhydrous dibasic calcium phosphate. In another subclass of this class, the diluent is microcrystalline cellulose and anhydrous dibasic calcium phosphate. In another subclass of this class, first diluent is anhydrous dibasic calcium phosphate and the second diluent is microcrystalline cellulose. In another subclass of this class, the diluent in the first layer is a mixture with a ratio of about 1:5 to about 1:6 of microcrystalline cellulose to anhydrous dibasic calcium phosphate; the disintegrant is croscarmellose sodium; and the lubricant is a mixture of magnesium stearate and sodium stearyl fumarate. In another subclass of this class, the diluent in the first layer is a mixture of 1:5.19 microcrystalline cellulose to anhydrous dibasic calcium phosphate; the disintegrant is croscarmellose sodium; and the lubricant is a mixture of magnesium stearate and sodium stearyl fumarate.

In another class of the embodiments of the present invention, the second layer of the pharmaceutical composition (the simvastatin layer) contains about 65-85% by weight of a diluent. In a subclass of this class, the composition contains about 70 to 80% by weight of a diluent. In another subclass of this class, the composition contains about 73 to 77% by weight of a diluent. In another subclass of this class, the composition contains about 75.73% by weight of a diluent. In another subclass of this class, the diluent is microcrystalline cellulose or lactose monohydrate or a mixture thereof. In another subclass of this class, the diluent is microcrystalline cellulose and lactose monohydrate.

In another class of the embodiments of the present invention, the first layer of the pharmaceutical composition (the sitagliptin layer) contains about 0.5-6% by weight of a disintegrant. In a subclass of this class, the composition contains about 1 to 4% of a disintegrant. In another subclass of this class, the composition contains about 1 to 3% of a disintegrant. In another subclass of this class, the composition contains about 2% of a disintegrant. In another subclass of this class, the disintegrant is croscarmellose sodium.

In another class of the embodiments of the present invention, the second layer of the pharmaceutical composition (the simvastatin layer) contains about 1-10% by weight of an anti-oxidant. In a subclass of this class, the composition contains about 2 to 5% by weight of an anti-oxidant. In another subclass of this class, the composition contains about 3 to 4% by weight of an anti-oxidant. In another subclass of this class, the composition contains about 3.77% by weight of an anti-oxidant. In another subclass of this class, the anti-oxidant is butylated hydroxyanisole, citric acid, citric acid monohydrate or ascorbic acid, or a mixture thereof. In another subclass of this class, the anti-oxidant is a mixture of butylated hydroxyanisole, citric acid or citric acid monohydrate and ascorbic acid.

In another class of the embodiments of the present invention, the first layer of the pharmaceutical composition (the sitagliptin layer) contains about 0.75 to 10% by weight of a lubricant. In a subclass of this class, the composition contains about 1.5 to 7% of a lubricant. In another subclass of this class, the composition contains about 4% of a lubricant. In another subclass of this class, the composition contains about 0.25 to 4% of a first lubricant; and contains about 0.5 to 6% of a second lubricant. In another subclass of this class, the composition contains about 2.5 to 3.5% of a first lubricant; and contains about 0.5 to 1.5% of a second lubricant. In another subclass of this class, the composition contains about 3% of a first lubricant; and contains about 1% of a second lubricant. In another subclass of this class, the lubricant is a mixture of sodium stearyl fumarate or magnesium stearate. In another subclass of this class, the lubricant is sodium stearyl fumarate and magnesium stearate. In another subclass of this class, the first lubricant is sodium stearyl fumarate. In another subclass of this class, the second lubricant is magnesium stearate.

In another class of the embodiments of the present invention, the second layer of the pharmaceutical composition (the simvastatin layer) contains about 0.1-1.5% by weight of a lubricant. In a subclass of this class, the composition contains about 0.1 to 1.0% by weight of a lubricant. In a subclass of this class, the composition contains about 0.25 to 0.75% by weight of a lubricant. In another subclass of this class, the composition contains about 0.5% by weight of a lubricant. In another subclass of this class, the lubricant is sodium stearyl fumarate or magnesium stearate. In another subclass of this class, the lubricant is a mixture of sodium stearyl fumarate and magnesium stearate. In another subclass of this class, the lubricant is sodium stearyl fumarate. In another subclass of this class, the lubricant is magnesium stearate.

In another class of the embodiments of the present invention, the second layer of the pharmaceutical composition (the simvastatin layer) contains about 5-15% by weight of a binding agent. In a subclass of this class, the composition contains about 8 to 12% by weight of a binding agent. In another subclass of this class, the composition contains about 9 to 11% by weight of a binding agent. In another subclass of this class, the composition contains about 10% by weight of a binding agent. In another subclass of this class, the binding agent is hydroxypropylcellulose, polyvinylpyrrolidone, pregelatinized starch or pregelatinized corn starch, or a mixture thereof. In another subclass of this class, the binding agent is pregelatinized starch or pregelatinized corn starch, or a mixture thereof. In another subclass of this class, the binding agent is pregelatinized starch. In another subclass of this class, the binding agent is pregelatinized corn starch.

In further embodiments of the present invention, the pharmaceutical compositions are envisioned for commercial development:

### Tablets of 50 mg Dipeptidyl Peptidase-4 Inhibitor/5 mg Simvastatin Potency

For the first layer: about 32.12% by weight of the first layer of the dipeptidyl peptidase-4 inhibitor or a pharmaceutically acceptable salt thereof; about 61-62% by weight of the first layer of a diluent; about 2-3% by weight of the first layer of a disintegrant; and about 4% by weight of the first layer of a lubricant. For the second layer: about 10% by weight of the second layer of simvastatin, about 75 to 76% by weight of the second layer of a diluent, about 3 to 4% by weight of the second layer of an anti-oxidant; about 0.1-1.0% by weight of the second layer of a lubricant; and about 10% by weight of the second layer of a binding agent. In a class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent is selected from the group consisting of: dibasic anhydrous calcium phosphate, microcrystalline cellulose, lactose monohydrate, or a mixture thereof; the disintegrant is sodium croscarmellose; the lubricant is selected from the group consisting of: magnesium stearate and sodium stearyl fumarate, or a mixture thereof; the anti-oxidant is selected from butylated hydroxyanisole, citric acid, citric acid monohydrate, and ascorbic acid, or a mixture thereof; and the binding agent is pregelatinized starch or pregelatinized corn starch. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the first layer is a mixture with a ratio of about 1:5 to about 1:6 of microcrystalline cellulose to anhydrous dibasic calcium phosphate; the disintegrant is croscarmellose sodium; and the lubricant is a mixture of magnesium stearate and sodium stearyl fumarate. In another class of this embodiment, the diluent in the first layer is a mixture of 1:5.19 microcrystalline cellulose to anhydrous dibasic calcium phosphate; the disintegrant is croscarmellose sodium; and the lubricant is a mixture of magnesium stearate and sodium stearyl fumarate. In another class of this embodiment, the diluent in the second layer is a mixture of lactose monohydrate and microcrystalline cellulose; the anti-oxidant in the second layer is a mixture of butylated hydroxyanisole, citric acid and/or citric acid monohydrate, and ascorbic acid; the lubricant in the second layer is magnesium stearate; and the binding agent in the second layer is pregelatinized starch and/or pregelatinized corn starch. In another subclass the dipeptidyl peptidase-4 inhibitor is sitagliptin, or a pharmaceutically acceptable salt thereof.

### Tablets of 50 mg Dipeptidyl Peptidase-4 Inhibitor/10 mg Simvastatin Potency

For the first layer: about 32.12% by weight of the first layer of the dipeptidyl peptidase-4 inhibitor or a pharmaceutically acceptable salt thereof; about 61-62% by weight of the first layer of a diluent; about 2-3% by weight of the first layer of a disintegrant; and about 4% by weight of the first layer of a lubricant. For the second layer: about 10% by weight of the second layer of simvastatin, about 75 to 76% by weight of the second layer of a diluent, about 3 to 4% by weight of the second layer of an anti-oxidant; about 0.1-1.0% by weight of the second layer of a lubricant; and about 10% by weight of the second layer of a binding agent. In a class of this embodiment, the dipeptidyl peptidase-4 inhibitor is selected from the group consisting of alogliptin, carmegliptin, melogliptin, dutogliptin, denagliptin, linagliptin, saxagliptin and vildagliptin, or a pharmaceutically acceptable salt thereof. In another class of this embodiment, the diluent is selected from the group consisting of: dibasic anhydrous calcium phosphate, microcrystalline cellulose, lactose monohydrate, or a mixture thereof; the disintegrant is sodium croscarmellose; the lubricant is selected from the group consisting of: magnesium stearate and sodium stearyl fumarate, or a mixture thereof; the anti-oxidant is selected from butylated hydroxyanisole, citric acid, citric acid monohydrate, and ascorbic acid, or a mixture thereof; and the binding agent is pregelatinized starch or pregelatinized corn starch. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the first layer is a mixture with a ratio of about 1:5 to about 1:6 of microcrystalline cellulose to anhydrous dibasic calcium phosphate; the disintegrant is croscarmellose sodium; and the lubricant is a mixture of magnesium stearate and sodium stearyl fumarate. In another class of this embodiment, the diluent in the first layer is a mixture of 1:5.19 microcrystalline cellulose to anhydrous dibasic calcium phosphate; the disintegrant is croscarmellose sodium; and the lubricant is a mixture of magnesium stearate and sodium stearyl fumarate. In another class of this embodiment, the diluent in the second layer is a mixture of lactose monohydrate and microcrystalline cellulose; the anti-oxidant in the second layer is a mixture of butylated hydroxyanisole, citric acid and/or citric acid monohydrate, and ascorbic acid; the lubricant in the second layer is magnesium stearate; and the binding agent in the second layer is pregelatinized starch and/or pregelatinized corn starch. In another subclass the dipeptidyl peptidase-4 inhibitor is sitagliptin, or a pharmaceutically acceptable salt thereof.

### Tablets of 50 mg Dipeptidyl Peptidase-4 Inhibitor/20 mg Simvastatin Potency

For the first layer: about 32.12% by weight of the first layer of the dipeptidyl peptidase-4 inhibitor or a pharmaceutically acceptable salt thereof; about 61-62% by weight of the first layer of a diluent; about 2-3% by weight of the first layer of a disintegrant; and about 4% by weight of the first layer of a lubricant. For the second layer: about 10% by weight of the second layer of simvastatin, about 75 to 76% by weight of the second layer of a diluent, about 3 to 4% by weight of the second layer of an anti-oxidant; about 0.1-1.0% by weight of the second layer of a lubricant; and about 10% by weight of the second layer of a binding agent. In a class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent is selected from the group consisting of: dibasic anhydrous calcium phosphate, microcrystalline cellulose, lactose monohydrate, or a mixture thereof; the disintegrant is sodium croscarmellose; the lubricant is selected from the group consisting of: magnesium stearate and sodium stearyl fumarate, or a mixture thereof; the anti-oxidant is selected from butylated hydroxyanisole, citric acid, citric acid monohydrate, and ascorbic acid, or a mixture thereof; and the binding agent is pregelatinized starch or pregelatinized corn starch. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the first layer is a mixture with a ratio of about 1:5 to about 1:6 of microcrystalline cellulose to anhydrous dibasic calcium phosphate; the disintegrant is croscarmellose sodium; and the lubricant is a mixture of magnesium stearate and sodium stearyl fumarate. In another class of this embodiment, the diluent in the first layer is a mixture of 1:5.19 microcrystalline cellulose to anhydrous dibasic calcium phosphate; the disintegrant is croscarmellose sodium; and the lubricant is a mixture of magnesium stearate and sodium stearyl fumarate. In another class of this embodiment, the diluent in the second layer is a mixture of lactose monohydrate and microcrystalline cellulose; the anti-oxidant in the second layer is a mixture of butylated hydroxyanisole, citric acid and/or citric acid monohydrate, and ascorbic acid; the lubricant in the second layer is magnesium stearate; and the binding agent in the second layer is pregelatinized starch and/or pregelatinized corn starch. In another subclass the dipeptidyl peptidase-4 inhibitor is sitagliptin, or a pharmaceutically acceptable salt thereof.

### Tablets of 50 mg Dipeptidylpeptidase-4 Inhibitor/40 mg Simvastatin Potency

For the first layer: about 32.12% by weight of the first layer of the dipeptidyl peptidase-4 inhibitor or a pharmaceutically acceptable salt thereof; about 61-62% by weight of the first layer of a diluent; about 2-3% by weight of the first layer of a disintegrant; and about 4% by weight of the first layer of a lubricant. For the second layer: about 10% by weight of the second layer of simvastatin, about 75 to 76% by weight of the second layer of a diluent, about 3 to 4% by weight of the second layer of an anti-oxidant; about 0.1-1.0% by weight of the second layer of a lubricant; and about 10% by weight of the second layer of a binding agent. In a class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent is selected from the group consisting of: dibasic anhydrous calcium phosphate, microcrystalline cellulose, lactose monohydrate, or a mixture thereof; the disintegrant is sodium croscarmellose; the lubricant is selected from the group consisting of: magnesium stearate and sodium stearyl fumarate, or a mixture thereof; the anti-oxidant is selected from butylated hydroxyanisole, citric acid, citric acid monohydrate, and ascorbic acid, or a mixture thereof; and the binding agent is pregelatinized starch or pregelatinized corn starch. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the first layer is a mixture with a ratio of about 1:5 to about 1:6 of microcrystalline cellulose to anhydrous dibasic calcium phosphate; the disintegrant is croscarmellose sodium; and the lubricant is a mixture of magnesium stearate and sodium stearyl fumarate. In another class of this embodiment, the diluent in the first layer is a mixture of 1:5.19 microcrystalline cellulose to anhydrous dibasic calcium phosphate; the disintegrant is croscarmellose sodium; and the lubricant is a mixture of magnesium stearate and sodium stearyl fumarate. In another class of this embodiment, the diluent in the second layer is a mixture of lactose monohydrate and microcrystalline cellulose; the anti-oxidant in the second layer is a mixture of butylated hydroxyanisole, citric acid and/or citric acid monohydrate, and ascorbic acid; the lubricant in the second layer is magnesium stearate; and the binding agent in the second layer is pregelatinized starch and/or pregelatinized corn starch. In another subclass the dipeptidyl peptidase-4 inhibitor is sitagliptin, or a pharmaceutically acceptable salt thereof.

### Tablets of 50 mg Dipeptidyl Peptidase-4 Inhibitor/80 mg Simvastatin Potency:

For the first layer: about 32.12% by weight of the first layer of the dipeptidyl peptidase-4 inhibitor or a pharmaceutically acceptable salt thereof; about 61-62% by weight of the first layer of a diluent; about 2-3% by weight of the first layer of a disintegrant; and about 4% by weight of the first layer of a lubricant. For the second layer: about 10% by weight of the second layer of simvastatin, about 75 to 76% by weight of the second layer of a diluent, about 3 to 4% by weight of the second layer of an anti-oxidant; about 0.1-1.0% by weight of the second layer of a lubricant; and about 10% by weight of the second layer of a binding agent. In a class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent is selected from the group consisting of: dibasic anhydrous calcium phosphate, microcrystalline cellulose, lactose monohydrate, or a mixture thereof; the disintegrant is sodium croscarmellose; the lubricant is selected from the group consisting of: magnesium stearate and sodium stearyl fumarate, or a mixture thereof; the anti-oxidant is selected from butylated hydroxyanisole, citric acid, citric acid monohydrate, and ascorbic acid, or a mixture thereof; and the binding agent is pregelatinized starch or pregelatinized corn starch. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the first layer is a mixture with a ratio of about 1:5 to about 1:6 of microcrystalline cellulose to anhydrous dibasic calcium phosphate; the disintegrant is croscarmellose sodium; and the lubricant is a mixture of magnesium stearate and sodium stearyl fumarate. In another class of this embodiment, the diluent in the first layer is a mixture of 1:5.19 microcrystalline cellulose to anhydrous dibasic calcium phosphate; the disintegrant is croscarmellose sodium; and the lubricant is a mixture of magnesium stearate and sodium stearyl fumarate. In another class of this embodiment, the diluent in the second layer is a mixture of lactose monohydrate and microcrystalline cellulose; the anti-oxidant in the second layer is a mixture of butylated hydroxyanisole, citric acid and/or citric acid monohydrate, and ascorbic acid; the lubricant in the second layer is magnesium stearate; and the binding agent in the second layer is pregelatinized starch and/or pregelatinized corn starch. In another subclass the dipeptidyl peptidase-4 inhibitor is sitagliptin, or a pharmaceutically acceptable salt thereof.

### Tablets of 100 mg Dipeptidyl Peptidase-4 Inhibitor/5 mg Simvastatin Potency:

For the first layer: about 32.12% by weight of the first layer of the dipeptidyl peptidase-4 inhibitor or a pharmaceutically acceptable salt thereof; about 61-62% by weight of the first layer of a diluent; about 2-3% by weight of the first layer of a disintegrant; and about 4% by weight of the first layer of a lubricant. For the second layer: about 10% by weight of the second layer of simvastatin, about 75 to 76% by weight of the second layer of a diluent, about 3 to 4% by weight of the second layer of an anti-oxidant; about 0.1-1.0% by weight of the second layer of a lubricant; and about 10% by weight of the second layer of a binding agent. In a class of this embodiment, the dipeptidyl peptidase-4 inhibitor is selected from the group consisting of alogliptin, carmegliptin, melogliptin, dutogliptin, denagliptin, linagliptin, saxagliptin and vildagliptin, or a pharmaceutically acceptable salt thereof. In another class of this embodiment, the diluent is selected from the group consisting of dibasic anhydrous calcium phosphate, microcrystalline cellulose, lactose monohydrate, or a mixture thereof; the disintegrant is sodium croscarmellose; the lubricant is selected from the group consisting of: magnesium stearate and sodium stearyl fumarate, or a mixture thereof; the anti-oxidant is selected from butylated hydroxyanisole, citric acid, citric acid monohydrate, and ascorbic acid, or a mixture thereof; and the binding agent is pregelatinized starch or pregelatinized corn starch. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the first layer is a mixture with a ratio of about 1:5 to about 1:6 of microcrystalline cellulose to anhydrous dibasic calcium phosphate; the disintegrant is croscarmellose sodium; and the lubricant is a mixture of magnesium stearate and sodium stearyl fumarate. In another class of this embodiment, the diluent in the first layer is a mixture of 1:5.19 microcrystalline cellulose to anhydrous dibasic calcium phosphate; the disintegrant is croscarmellose sodium; and the lubricant is a mixture of magnesium stearate and sodium stearyl fumarate. In another class of this embodiment, the diluent in the second layer is a mixture of lactose monohydrate and microcrystalline cellulose; the anti-oxidant in the second layer is a mixture of butylated hydroxyanisole, citric acid and/or citric acid monohydrate, and ascorbic acid; the lubricant in the second layer is magnesium stearate; and the binding agent in the second layer is pregelatinized starch and/or pregelatinized corn starch. In another subclass the dipeptidyl peptidase-4 inhibitor is sitagliptin, or a pharmaceutically acceptable salt thereof.

### Tablets of 100 mg Dipeptidyl Peptidase-4 Inhibitor/10 mg Simvastatin Potency:

For the first layer: about 32.12% by weight of the first layer of the dipeptidyl peptidase-4 inhibitor or a pharmaceutically acceptable salt thereof; about 61-62% by weight of the first layer of a diluent; about 2-3% by weight of the first layer of a disintegrant; and about 4% by weight of the first layer of a lubricant. For the second layer: about 10% by weight of the second layer of simvastatin, about 75 to 76% by weight of the second layer of a diluent, about 3 to 4% by weight of the second layer of an anti-oxidant; about 0.1-1.0% by weight of the second layer of a lubricant; and about 10% by weight of the second layer of a binding agent. In a class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent is selected from the group consisting of: dibasic anhydrous calcium phosphate, microcrystalline cellulose, lactose monohydrate, or a mixture thereof; the disintegrant is sodium croscarmellose; the lubricant is selected from the group consisting of: magnesium stearate and sodium stearyl fumarate, or a mixture thereof; the anti-oxidant is selected from butylated hydroxyanisole, citric acid, citric acid monohydrate, and ascorbic acid, or a mixture thereof; and the binding agent is pregelatinized starch or pregelatinized corn starch. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the first layer is a mixture with a ratio of about 1:5 to about 1:6 of microcrystalline cellulose to anhydrous dibasic calcium phosphate; the disintegrant is croscarmellose sodium; and the lubricant is a mixture of magnesium stearate and sodium stearyl fumarate. In another class of this embodiment, the diluent in the first layer is a mixture of 1:5.19 microcrystalline cellulose to anhydrous dibasic calcium phosphate; the disintegrant is croscarmellose sodium; and the lubricant is a mixture of magnesium stearate and sodium stearyl fumarate. In another class of this embodiment, the diluent in the second layer is a mixture of lactose monohydrate and microcrystalline cellulose; the anti-oxidant in the second layer is a mixture of butylated hydroxyanisole, citric acid and/or citric acid monohydrate, and ascorbic acid; the lubricant in the second layer is magnesium stearate; and the binding agent in the second layer is pregelatinized starch and/or pregelatinized corn starch. In another subclass the dipeptidyl peptidase-4 inhibitor is sitagliptin, or a pharmaceutically acceptable salt thereof.

### Tablets of 100 mg Dipeptidyl Peptidase-4 Inhibitor/20 mg Simvastatin Potency:

For the first layer: about 32.12% by weight of the first layer of the dipeptidyl peptidase-4 inhibitor or a pharmaceutically acceptable salt thereof; about 61-62% by weight of the first layer of a diluent; about 2-3% by weight of the first layer of a disintegrant; and about 4% by weight of the first layer of a lubricant. For the second layer: about 10% by weight of the second layer of simvastatin, about 75 to 76% by weight of the second layer of a diluent, about 3 to 4% by weight of the second layer of an anti-oxidant; about 0.1-1.0% by weight of the second layer of a lubricant; and about 10% by weight of the second layer of a binding agent. In a class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent is selected from the group consisting of: dibasic anhydrous calcium phosphate, microcrystalline cellulose, lactose monohydrate, or a mixture thereof; the disintegrant is sodium croscarmellose; the lubricant is selected from the group consisting of: magnesium stearate and sodium stearyl fumarate, or a mixture thereof; the anti-oxidant is selected from butylated hydroxyanisole, citric acid, citric acid monohydrate, and ascorbic acid, or a mixture thereof; and the binding agent is pregelatinized starch or pregelatinized corn starch. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the first layer is a mixture with a ratio of about 1:5 to about 1:6 of microcrystalline cellulose to anhydrous dibasic calcium phosphate; the disintegrant is croscarmellose sodium; and the lubricant is a mixture of magnesium stearate and sodium stearyl fumarate. In another class of this embodiment, the diluent in the first layer is a mixture of 1:5.19 microcrystalline cellulose to anhydrous dibasic calcium phosphate; the disintegrant is croscarmellose sodium; and the lubricant is a mixture of magnesium stearate and sodium stearyl fumarate. In another class of this embodiment, the diluent in the second layer is a mixture of lactose monohydrate and microcrystalline cellulose; the anti-oxidant in the second layer is a mixture of butylated hydroxyanisole, citric acid and/or citric acid monohydrate, and ascorbic acid; the lubricant in the second layer is magnesium stearate; and the binding agent in the second layer is pregelatinized starch and/or pregelatinized corn starch. In another subclass the dipeptidyl peptidase-4 inhibitor is sitagliptin, or a pharmaceutically acceptable salt thereof.

### Tablets of 100 mg Dipeptidyl Peptidase-4 Inhibitor/40 mg Simvastatin Potency:

For the first layer: about 32.12% by weight of the first layer of the dipeptidyl peptidase-4 inhibitor or a pharmaceutically acceptable salt thereof; about 61-62% by weight of the first layer of a diluent; about 2-3% by weight of the first layer of a disintegrant; and about 4% by weight of the first layer of a lubricant. For the second layer: about 10% by weight of the second layer of simvastatin, about 75 to 76% by weight of the second layer of a diluent, about 3 to 4% by weight of the second layer of an anti-oxidant; about 0.1-1.0% by weight of the second layer of a lubricant; and about 10% by weight of the second layer of a binding agent. In a class of this embodiment, the dipeptidyl peptidase-4 inhibitor is selected from the group consisting of alogliptin, carmegliptin, melogliptin, dutogliptin, denagliptin, linagliptin, saxagliptin and vildagliptin, or a pharmaceutically acceptable salt thereof. In another class of this embodiment, the diluent is selected from the group consisting of: dibasic anhydrous calcium phosphate, microcrystalline cellulose, lactose monohydrate, or a mixture thereof; the disintegrant is sodium croscarmellose; the lubricant is selected from the group consisting of: magnesium stearate and sodium stearyl fumarate, or a mixture thereof; the anti-oxidant is selected from butylated hydroxyanisole, citric acid, citric acid monohydrate, and ascorbic acid, or a mixture thereof; and the binding agent is pregelatinized starch or pregelatinized corn starch. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the first layer is a mixture with a ratio of about 1:5 to about 1:6 of microcrystalline cellulose to anhydrous dibasic calcium phosphate; the disintegrant is croscarmellose sodium; and the lubricant is a mixture of magnesium stearate and sodium stearyl fumarate. In another class of this embodiment, the diluent in the first layer is a mixture of 1:5.19 microcrystalline cellulose to anhydrous dibasic calcium phosphate; the disintegrant is croscarmellose sodium; and the lubricant is a mixture of magnesium stearate and sodium stearyl fumarate. In another class of this embodiment, the diluent in the second layer is a mixture of lactose monohydrate and microcrystalline cellulose; the anti-oxidant in the second layer is a mixture of butylated hydroxyanisole, citric acid and/or citric acid monohydrate, and ascorbic acid; the lubricant in the second layer is magnesium stearate; and the binding agent in the second layer is pregelatinized starch and/or pregelatinized corn starch. In another subclass the dipeptidyl peptidase-4 inhibitor is sitagliptin, or a pharmaceutically acceptable salt thereof.

### Tablets of 100 mg Dipeptidyl Peptidase-4 Inhibitor/80 mg Simvastatin Potency:

For the first layer: about 32.12% by weight of the first layer of the dipeptidyl peptidase-4 inhibitor or a pharmaceutically acceptable salt thereof; about 61-62% by weight of the first layer of a diluent; about 2-3% by weight of the first layer of a disintegrant; and about 4% by weight of the first layer of a lubricant. For the second layer: about 10% by weight of the second layer of simvastatin, about 75 to 76% by weight of the second layer of a diluent, about 3 to 4% by weight of the second layer of an anti-oxidant; about 0.1-1.0% by weight of the second layer of a lubricant; and about 10% by weight of the second layer of a binding agent. In a class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent is selected from the group consisting of: dibasic anhydrous calcium phosphate, microcrystalline cellulose, lactose monohydrate, or a mixture thereof; the disintegrant is sodium croscarmellose; the lubricant is selected from the group consisting of: magnesium stearate and sodium stearyl fumarate, or a mixture thereof; the anti-oxidant is selected from butylated hydroxyanisole, citric acid, citric acid monohydrate, and ascorbic acid, or a mixture thereof; and the binding agent is pregelatinized starch or pregelatinized corn starch. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the first layer is a mixture with a ratio of about 1:5 to about 1:6 of microcrystalline cellulose to anhydrous dibasic calcium phosphate; the disintegrant is croscarmellose sodium; and the lubricant is a mixture of magnesium stearate and sodium stearyl fumarate. In another class of this embodiment, the diluent in the first layer is a mixture of 1:5.19 microcrystalline cellulose to anhydrous dibasic calcium phosphate; the disintegrant is croscarmellose sodium; and the lubricant is a mixture of magnesium stearate and sodium stearyl fumarate. In another class of this embodiment, the diluent in the second layer is a mixture of lactose monohydrate and microcrystalline cellulose; the anti-oxidant in the second layer is a mixture of butylated hydroxyanisole, citric acid and/or citric acid monohydrate, and ascorbic acid; the lubricant in the second layer is magnesium stearate; and the binding agent in the second layer is pregelatinized starch and/or pregelatinized corn starch. In another subclass the dipeptidyl peptidase-4 inhibitor is sitagliptin, or a pharmaceutically acceptable salt thereof.

The pharmaceutical tablet compositions of the present invention may also contain one or more additional formulation ingredients selected from a wide variety of excipients known in the pharmaceutical formulation art. According to the desired properties of the pharmaceutical composition, any number of ingredients may be selected, alone or in combination, based upon their known uses in preparing tablet compositions. Such ingredients include, but are not limited to, diluents, compression aids, glidants, disintegrants, lubricants, flavors, flavor enhancers, sweeteners, and preservatives.

The term “tablet” as used herein is intended to encompass compressed pharmaceutical dosage formulations of all shapes and sizes, whether coated or uncoated. Substances which may be used for coating include hydroxypropylcellulose, hydroxypropylmethylcellulose, titanium dioxide, talc, sweeteners, colorants, and flavoring agents.

The term and symbol “% by weight” and “%” as used herein refers to the percentage by weight of the excipient and active ingredient (DPP-4 inhibitor or simvastatin) in each individual layer in the bilayer tablet, wherein the “individual layer” means the first layer or the second layer of the bilayer tablet.

In one embodiment the pharmaceutical compositions of the present invention are prepared by wet granulation (simvastatin layer) and dry processing (DPP-4 inhibitor layer). In one class of this embodiment, the simvastatin layer was prepared by fluid bed wet granulation. In another class of this embodiment, the simvastatin layer was prepared by high shear granulation. In another class of this embodiment, the simvastatin layer was prepared by high shear wet granulation. In another class of this embodiment, the DPP-4 layer was prepared by direct compression. In another class of this embodiment, the DPP-4 layer was prepared by dry direct compression. Granulation is a process in which binding agent is added either through the granulating solution or through addition to the granulating bowl to form granules. The steps involved in the wet granulation and dry processing method comprise the following:

(1) Preparation of the first layer containing Sitagliptin phosphate:
(i) Sitagliptin phosphate, microcrystalline cellulose, croscarmellose sodium, and dibasic calcium phosphate (anhydrous) are blended in a suitable blender to form a blend;
(ii) the above blend is lubricated with at least one lubricant (such as magnesium stearate and/or sodium stearyl fumarate) in a suitable blender to form a lubricated powder blend;
   (2) Preparation of the second layer containing simvastatin:
(i) simvastatin (containing 0.01% butylated hydroxyanisole), lactose, cellulose microcrystalline, and pregelatinized starch are mixed by the addition of a solution of butylated hydroxyanisole, ascorbic acid and citric acid to give a wet milled granulation;
(ii) the wet milled granulation is dried and milled;
(iii) the milled granulation is mixed with a lubricant, such as magnesium stearate to give a lubricated simvastatin granulation blend.
   (3) Preparation of the Bilayer Tablet via bilayer compression and coating:
(i) the sitagliptin layer is added to the bilayer press and tamped lightly;
(ii) the simvastatin layer is added and main compression force is applied to give bilayer tablets; and
(iii) the bilayer tablets are coated with a suitable coating.

The first layer (the DPP-4 inhibitor or sitagliptin layer) can be the layer at the bottom of the bilayer tablet or at the top of the bilayer tablet (filled into the die either first or second). The second layer (the simvastatin layer) can be the layer at the bottom of the bilayer tablet or at the top of the bilayer tablet (filled into the die either first or second).

The present invention provides a fixed dose combination of a dipeptidyl peptidase-4 (DPP-4) inhibitor, including but not limited to sitagliptin, or a pharmaceutically acceptable salt thereof, and simvastatin, or a pharmaceutically acceptable salt thereof, in which both drugs are stable in a single tablet. More particularly, the present invention provides a fixed dose combination comprised of a layer of a dipeptidyl peptidase-4 (DPP-4) inhibitor, including but not limited to sitagliptin, or a pharmaceutically acceptable salt thereof, and a layer of simvastatin in a single bilayer tablet.

The present invention also provides methods for treating Type 2 diabetes and hypercholesterolemia by orally administering to a host in need of such treatment a therapeutically effective amount of one of the fixed-dose combination pharmaceutical compositions of the present invention. In one embodiment the patient in need of such treatment is a human. In another embodiment the pharmaceutical composition is in the dosage form of a tablet. The pharmaceutical compositions comprising the fixed-dose combination may be administered once-daily (QD), twice-daily (BID), or thrice-daily (TID).

The following examples further describe and demonstrate embodiments within the scope of the present invention. The examples are given solely for the purpose of illustration and are not intended to be construed as limitations of the present invention as many variations thereof are possible without departing from the spirit and scope of the invention.

### Example 1

### Fixed-Dose Combination of 100 Milligrams Sitagliptin and 10 Milligrams Simvastatin Per Bilayer Tablet

| | |
|---|---|
| Dose (sitagliptin/simvastatin, mg/mg) | 100/10 |

Sitagliptin - 1^{st} Layer of Bilayer Tablet (mg/tablet)

| | |
|---|---|
| Sitagliptin phosphate monohydrate | 128.5¹ |
| Calcium Phosphate, Dibasic, Anhydrous | 207.5 |
| Microcrystalline Cellulose | 40.00 |
| Croscarmellose Sodium | 8.000 |
| Sodium Stearyl Fumarate | 12.00 |
| Magnesium Stearate | 4.000 |
| Total sitagliptin layer weight | 400 |

Simvastatin - 2^{nd} layer of Bilayer Tablet (mg/tablet)

| | |
|---|---|
| Simvastatin, [0.01% BHA] | 10.00 |
| Butylated hydroxyanisole (BHA) | 0.020 |
| Ascorbic acid | 2.500 |
| Citric acid monohydrate | 1.250 |
| Lactose, monohydrate | 70.73 |
| Microcrystalline cellulose | 5.000 |
| Pregelatinized corn starch | 10.00 |
| Magnesium stearate | 0.500 |
| Industrial Methylated Spirit 74OP^{,3,2} | — |
| Water purified, USP^{3,2} | — |
| Total simvastatin layer weight | 100 |

Film coating

| | |
|---|---|
| Purple Opadry II [85F170000] | 18.31 |
| Water, purified USP^{2,3} | — |
| Total Final Tablet Weight | 518.3 |

¹equivalent to 100 mg of free base with conversion factor of 1.285.
²Removed during processing
³Not included in total

### Method of Manufacture:

### Preparation of the Layer Containing Sitagliptin (the Sitagliptin Granulation Blend Layer):

The specific quantities of microcrystalline cellulose, croscarmellose sodium, and dibasic calcium phosphate (anhydrous) were charged with sitagliptin phosphate monohydrate to a diffusion blender. The materials were blended via diffusion (tumble) mixing. Magnesium stearate and sodium stearyl fumarate (lubricants) were screened and added to the bin blender. The blend was lubricated via diffusion (tumble) mixing to give the lubricated sitagliptin granulation blend.

### Preparation of the Layer Containing Simvastatin (the Simvastatin Granulation Blend Layer):

Granulation of a well blended mixture of simvastatin (containing 0.01% butylated hydroxyanisole), lactose, cellulose microcrystalline, and pregelatinized starch was accomplished by the addition of a hydro-alcoholic solution composed of butylated hydroxyanisole, ascorbic acid, and citric acid. The wet milled granulation was then dried and milled. Then the specific quantities of dried, milled, unlubricated simvastatin granulation and magnesium stearate (which had been screened), were charged to a diffusion blender. The materials were lubricated via diffusion (tumble) mixing to give the lubricated simvastatin granulation blend.

### Compression and Formation of the Bilayer Tablet:

The specified quantities of feed materials (lubricated simvastatin granulation blend and lubricated sitagliptin granulation blend) were compressed into bilayer tablets with the specified markings on a rotary tablet press. During the film coating step, a film-coating suspension was prepared. The compressed tablets were loaded into a coating pan and film coated with a suitable Opadry® or Opadry® II suspension (such as Purple Opadry® II [85F 170000]) to provide the film coated tablet.

### Example 2

### Fixed-Dose Combination of 100 Milligrams Sitagliptin and 20 Milligrams Simvastatin Per Bilayer Tablet

| | |
|---|---|
| Dose (sitagliptin/simvastatin, mg/mg) | 100/20 |

Sitagliptin - 1^{st} Layer of Bilayer Tablet (mg/tablet)

| | |
|---|---|
| Sitagliptin | 128.5¹ |
| Calcium Phosphate, Dibasic, Anhydrous | 207.5 |
| Microcrystalline Cellulose | 40.00 |
| Croscarmellose Sodium | 8.000 |
| Sodium Stearyl Fumarate | 12.00 |
| Magnesium Stearate | 4.000 |
| Total sitagliptin layer weight | 400.0 |

Simvastatin - 2^{nd} layer of Bilayer Tablet (mg/tablet)

| | |
|---|---|
| Simvastatin, [0.01% BHA] | 20.00 |
| Butylated hydroxyanisole (BHA) | 0.040 |
| Ascorbic acid | 5.000 |
| Citric acid monohydrate | 2.500 |
| Lactose, monohydrate | 141.5 |
| Microcrystalline cellulose | 10.00 |
| Pregelatinized corn starch | 20.00 |
| Magnesium stearate | 1.000 |
| Industrial Methylated Spirit 74OP^{,3,2} | — |
| Water purified, USP^{3,2} | — |
| Total simvastatin layer weight | 200 |

Film coating

| | |
|---|---|
| Purple Opadry II [85F170000] | 22.41 |
| Water, purified USP^{2,3} | — |
| Total Final Tablet Weight | 622.4 |

¹equivalent to 100 mg of free base with conversion factor of 1.285.
²Removed during processing
³Not included in total

### Method of Manufacture:

### Preparation of the Layer Containing Sitagliptin (the Sitagliptin Granulation Blend Layer):

The specific quantities of microcrystalline cellulose, croscarmellose sodium, and dibasic calcium phosphate (anhydrous) were charged with sitagliptin phosphate monohydrate to a diffusion blender. The materials were blended via diffusion (tumble) mixing. Magnesium stearate and sodium stearyl fumarate (lubricants) were screened and added to the bin blender. The blend was lubricated via diffusion (tumble) mixing to give the lubricated sitagliptin granulation blend.

### Preparation of the Layer Containing Simvastatin (the Simvastatin Granulation Blend Layer):

Granulation of a well blended mixture of simvastatin (containing 0.01% butylated hydroxyanisole), lactose, cellulose microcrystalline, and pregelatinized starch was accomplished by the addition of a hydro-alcoholic solution composed of butylated hydroxyanisole, ascorbic acid, and citric acid. The wet milled granulation was then dried and milled. Then the specific quantities of dried, milled, unlubricated simvastatin granulation and magnesium stearate (which had been screened), were charged to a diffusion blender. The materials were lubricated via diffusion (tumble) mixing to give the lubricated simvastatin granulation blend.

### Compression and Formation of the Bilayer Tablet:

The specified quantities of feed materials (lubricated simvastatin granulation blend and lubricated sitagliptin granulation blend) were compressed into bilayer tablets with the specified markings on a rotary tablet press. During the film coating step, a film-coating suspension was prepared. The compressed tablets were loaded into a coating pan and film coated with a suitable Opadry® or Opadry® II suspension (such as Purple Opadry® II [85F170000]) to provide the film coated tablet.

### Example 3

### Fixed-Dose Combination of 100 Milligrams Sitagliptin and 40 Milligrams Simvastatin Per Bilayer Tablet

| | |
|---|---|
| Dose (sitagliptin/simvastatin, mg/mg) | 100/40 |

Sitagliptin - 1^{st} Layer of Bilayer Tablet (mg/tablet)

| | |
|---|---|
| Sitagliptin | 128.5¹ |
| Calcium Phosphate, Dibasic, Anhydrous | 207.5 |
| Microcrystalline Cellulose | 40.00 |
| Croscarmellose Sodium | 8.000 |
| Sodium Stearyl Fumarate | 12.00 |
| Magnesium Stearate | 4.000 |
| Total sitagliptin layer weight | 400 |

Simvastatin - 2^{nd} layer of Bilayer Tablet (mg/tablet)

| | |
|---|---|
| Simvastatin, [0.01% BHA] | 40.00 |
| Butylated hydroxyanisole (BHA) | 0.080 |
| Ascorbic acid | 10.00 |
| Citric acid monohydrate | 5.000 |
| Lactose, monohydrate | 282.9 |
| Microcrystalline cellulose | 20.00 |
| Pregelatinized corn starch | 40.00 |
| Magnesium stearate | 2.000 |
| Industrial Methylated Spirit 74OP^{,3,2} | — |
| Water purified, USP^{3,2} | — |
| Total simvastatin layer weight | 400 |

Film coating

| | |
|---|---|
| Beige Opadry [85F170001] | 27.59 |
| Water, purified USP^{2,3} | |
| Total Final Tablet Weight | 827.6 |

¹equivalent to 100 mg of free base with conversion factor of 1.285.
²Removed during processing
³Not included in total

### Method of Manufacture:

### Preparation of the Layer Containing Sitagliptin (the Sitagliptin Granulation Blend Layer):

The specific quantities of microcrystalline cellulose, croscarmellose sodium, and dibasic calcium phosphate (anhydrous) were charged with sitagliptin phosphate monohydrate to a diffusion blender. The materials were blended via diffusion (tumble) mixing. Magnesium stearate and sodium stearyl fumarate (lubricants) were screened and added to the bin blender. The blend was lubricated via diffusion (tumble) mixing to give the lubricated sitagliptin granulation blend.

### Preparation of the Layer Containing Simvastatin (the Simvastatin Granulation Blend Layer):

Granulation of a well blended mixture of simvastatin (containing 0.01% butylated hydroxyanisole), lactose, cellulose microcrystalline, and pregelatinized starch was accomplished by the addition of a hydro-alcoholic solution composed of butylated hydroxyanisole, ascorbic acid, and citric acid. The wet milled granulation was then dried and milled. Then the specific quantities of dried, milled, unlubricated simvastatin granulation and magnesium stearate (which had been screened), were charged to a diffusion blender. The materials were lubricated via diffusion (tumble) mixing to give the lubricated simvastatin granulation blend.

### Compression and Formation of the Bilayer Tablet:

The specified quantities of feed materials (lubricated simvastatin granulation blend and lubricated sitagliptin granulation blend) were compressed into bilayer tablets with the specified markings on a rotary tablet press. During the film coating step, a film-coating suspension was prepared. The compressed tablets were loaded into a coating pan and film coated with a suitable Opadry® or Opadry® II suspension (such as Beige Opadry [85F170001]) to provide the film coated tablet.

### Example 4

### Fixed-Dose Combination of 100 Milligrams Sitagliptin and 80 Milligrams Simvastatin Per Bilayer Tablet

| | |
|---|---|
| Dose (sitagliptin/simvastatin, mg/mg) | 100/80 |

Sitagliptin - 1^{st} Layer of Bilayer Tablet (mg/tablet)

| | |
|---|---|
| Sitagliptin phosphate | 128.5¹ |
| Calcium Phosphate, Dibasic, Anhydrous | 207.5 |
| Microcrystalline Cellulose | 40.00 |
| Croscarmellose Sodium | 8.000 |
| Sodium Stearyl Fumarate | 12.00 |
| Magnesium Stearate | 4.000 |
| Total sitagliptin layer weight | 400.0 |

Simvastatin - 2^{nd} layer of Bilayer Tablet (mg/tablet)

| | |
|---|---|
| Simvastatin, [0.01% BHA] | 80.00 |
| Butylated hydroxyanisole (BHA) | 0.160 |
| Ascorbic acid | 20.00 |
| Citric acid monohydrate | 10.00 |
| Lactose, monohydrate | 565.8 |
| Microcrystalline cellulose | 40.00 |
| Pregelatinized corn starch | 80.00 |
| Magnesium stearate | 4.00 |
| Industrial Methylated Spirit 74OP^{,3,2} | — |
| Water purified, USP^{3,2} | — |
| Total simvastatin layer weight | 800 |

Film coating

| | |
|---|---|
| Purple Opadry II [85F170000] | 36.04 |
| Water, purified USP^{2,3} | — |
| Total Final Tablet Weight | 1236 |

¹equivalent to 100 mg of free base with conversion factor of 1.285.
²Removed during processing
³Not included in total

### Method of Manufacture:

### Preparation of the Layer Containing Sitagliptin (the Sitagliptin Granulation Blend Layer):

The specific quantities of microcrystalline cellulose, croscarmellose sodium, and dibasic calcium phosphate (anhydrous) were charged with sitagliptin phosphate monohydrate to a diffusion blender. The materials were blended via diffusion (tumble) mixing. Magnesium stearate and sodium stearyl fumarate (lubricants) were screened and added to the bin blender. The blend was lubricated via diffusion (tumble) mixing to give the lubricated sitagliptin granulation blend.

### Preparation of the Layer Containing Simvastatin (the Simvastatin Granulation Blend Layer):

Granulation of a well blended mixture of simvastatin (containing 0.01% butylated hydroxyanisole), lactose, cellulose microcrystalline, and pregelatinized starch was accomplished by the addition of a hydro-alcoholic solution composed of butylated hydroxyanisole, ascorbic acid, and citric acid. The wet milled granulation was then dried and milled. Then the specific quantities of dried, milled, unlubricated simvastatin granulation and magnesium stearate (which had been screened), were charged to a diffusion blender. The materials were lubricated via diffusion (tumble) mixing to give the lubricated simvastatin granulation blend.

### Compression and Formation of the Bilayer Tablet:

The specified quantities of feed materials (lubricated simvastatin granulation blend and lubricated sitagliptin granulation blend) were compressed into bilayer tablets with the specified markings on a rotary tablet press. During the film coating step, a film-coating suspension was prepared. The compressed tablets were loaded into a coating pan and film coated with a suitable Opadry® or Opadry® II suspension (such as Purple Opadry® II [85F170000]) to provide the film coated tablet.

### Example 5

### Fixed-Dose Combination of 50 Milligrams Sitagliptin and 10 Milligrams Simvastatin Per Bilayer Tablet

| | |
|---|---|
| Dose (sitagliptin/simvastatin, mg/mg) | 50/10 |

Sitagliptin - 1^{st} Layer of Bilayer Tablet (mg/tablet)

| | |
|---|---|
| Sitagliptin phosphate | 64.26¹ |
| Dibasic calcium phosphate, anhydrous | 103.8 |
| Microcrystalline cellulose | 20.00 |
| Croscarmellose sodium | 4.000 |
| Sodium stearyl fumarate | 6.000 |
| Magnesium stearate | 2.000 |
| Total sitagliptin layer | 200.1 |

Simvastatin - 2^{nd} layer of Bilayer Tablet (mg/tablet)

| | |
|---|---|
| Simvastatin, [0.01% BHA] | 10.00 |
| Butylated hydroxyanisole (BHA) | 0.020 |
| Ascorbic acid | 2.500 |
| Citric acid monohydrate | 1.250 |
| Lactose monohydrate | 70.73 |
| Microcrystalline cellulose | 5.000 |
| Pregelatinized corn starch | 10.00 |
| Magnesium stearate | 0.500 |
| Industrial methylated spirit² | — |
| Water purified² | — |
| Total simvastatin layer weight | 100.0 |

Film coating

| | |
|---|---|
| OPADRY ® II Red³ | 14.15 |
| Water, purified² | — |
| Total Final Tablet Weight | 314.3 |

¹Equivalent to 50 mg of sitagliptin free base with conversion factor of 1.285.
²Removed during processing
³OPADRY Red and OPADRY Orange-Beige are based on the similar compositions of polyvinyl alcohol, talc, macrogrol/PEG and titanium dioxide.

### Method of Manufacture:

### Preparation of the Layer Containing Sitagliptin (the Sitagliptin Granulation Blend Layer):

The specific quantities of microcrystalline cellulose, croscarmellose sodium, and dibasic calcium phosphate (anhydrous) were charged with sitagliptin phosphate monohydrate to a diffusion blender. The materials were blended via diffusion (tumble) mixing. Magnesium stearate and sodium stearyl fumarate (lubricants) were screened and added to the bin blender. The blend was lubricated via diffusion (tumble) mixing to give the lubricated sitagliptin granulation blend.

### Preparation of the Layer Containing Simvastatin (the Simvastatin Granulation Blend Layer):

Granulation of a well blended mixture of simvastatin (containing 0.01% butylated hydroxyanisole), lactose, cellulose microcrystalline, and pregelatinized starch was accomplished by the addition of a hydro-alcoholic solution composed of butylated hydroxyanisole, ascorbic acid, and citric acid. The wet milled granulation was then dried and milled. Then the specific quantities of dried, milled, unlubricated simvastatin granulation and magnesium stearate (which had been screened), were charged to a diffusion blender. The materials were lubricated via diffusion (tumble) mixing to give the lubricated simvastatin granulation blend.

### Compression and Formation of the Bilayer Tablet:

The specified quantities of feed materials (lubricated simvastatin granulation blend and lubricated sitagliptin granulation blend) were compressed into bilayer tablets with the specified markings on a rotary tablet press. During the film coating step, a film-coating suspension was prepared. The compressed tablets were loaded into a coating pan and film coated with a suitable Opadry® or Opadry® II suspension (such as OPADRY® II Red) to provide the film coated tablet.

### Example 6

### Fixed-Dose Combination of 50 Milligrams Sitagliptin and 20 Milligrams Simvastatin Per Bilayer Tablet

| | |
|---|---|
| Dose (sitagliptin/simvastatin, mg/mg) | 50/20 |

Sitagliptin - 1^{st} Layer of Bilayer Tablet (mg/tablet)

| | |
|---|---|
| Sitagliptin phosphate | 64.26¹ |
| Dibasic calcium phosphate, anhydrous | 103.8 |
| Microcrystalline cellulose | 20.00 |
| Croscarmellose sodium | 4.000 |
| Sodium stearyl fumarate | 6.000 |
| Magnesium stearate | 2.000 |
| Total sitagliptin layer weight | 200.1 |

Simvastatin - 2^{nd} layer of Bilayer Tablet (mg/tablet)

| | |
|---|---|
| Simvastatin, [0.01% BHA] | 20.00 |
| Butylated hydroxyanisole (BHA) | 0.040 |
| Ascorbic acid | 5.000 |
| Citric acid monohydrate | 2.500 |
| Lactose monohydrate | 141.5 |
| Microcrystalline cellulose | 10.00 |
| Pregelatinized corn starch | 20.00 |
| Magnesium stearate | 1.000 |
| Industrial methylated spirit² | — |
| Water purified² | — |
| Total simvastatin layer weight | 200.0 |

Film coating

| | |
|---|---|
| OPADRY ® II Orange Beige³ | 17.49 |
| Water, purified² | — |
| 623.4 | 417.6 |

¹Equivalent to 50 mg of sitagliptin free base with conversion factor of 1.285.
²Removed during processing
³OPADRY Red and OPADRY Orange-Beige are based on the similar compositions of polyvinyl alcohol, talc, macrogrol/PEG and titanium dioxide.

### Method of Manufacture.

### Preparation of the Layer Containing Sitagliptin (the Sitagliptin Granulation Blend Layer):

The specific quantities of microcrystalline cellulose, croscarmellose sodium, and dibasic calcium phosphate (anhydrous) were charged with sitagliptin phosphate monohydrate to a diffusion blender. The materials were blended via diffusion (tumble) mixing. Magnesium stearate and sodium stearyl fumarate (lubricants) were screened and added to the bin blender. The blend was lubricated via diffusion (tumble) mixing to give the lubricated sitagliptin granulation blend.

### Preparation of the Layer Containing Simvastatin (the Simvastatin Granulation Blend Layer):

Granulation of a well blended mixture of simvastatin (containing 0.01% butylated hydroxyanisole), lactose, cellulose microcrystalline, and pregelatinized starch was accomplished by the addition of a hydro-alcoholic solution composed of butylated hydroxyanisole, ascorbic acid, and citric acid. The wet milled granulation was then dried and milled. Then the specific quantities of dried, milled, unlubricated simvastatin granulation and magnesium stearate (which had been screened), were charged to a diffusion blender. The materials were lubricated via diffusion (tumble) mixing to give the lubricated simvastatin granulation blend.

### Compression and Formation of the Bilayer Tablet:

The specified quantities of feed materials (lubricated simvastatin granulation blend and lubricated sitagliptin granulation blend) were compressed into bilayer tablets with the specified markings on a rotary tablet press. During the film coating step, a film-coating suspension was prepared. The compressed tablets were loaded into a coating pan and film coated with a suitable Opadry® or Opadry® II suspension (such as OPADRY® II Orange Beige) to provide the film coated tablet.

### Example 7

### Fixed-Dose Combination of 50 Milligrams Sitagliptin and 40 Milligrams Simvastatin Per Bilayer Tablet

| | |
|---|---|
| Dose (sitagliptin/simvastatin, mg/mg) | 50/40 |

Sitagliptin - 1^{st} Layer of Bilayer Tablet (mg/tablet)

| | |
|---|---|
| Sitagliptin phosphate | 64.26¹ |
| Dibasic calcium phosphate, anhydrous | 103.8 |
| Microcrystalline cellulose | 20.00 |
| Croscarmellose sodium | 4.000 |
| Sodium stearyl fumarate | 6.000 |
| Magnesium stearate | 2.000 |
| Total sitagliptin layer weight | 200.1 |

Simvastatin - 2^{nd} layer of Bilayer Tablet (mg/tablet)

| | |
|---|---|
| Simvastatin, [0.01% BHA] | 40.00 |
| Butylated hydroxyanisole (BHA) | 0.080 |
| Ascorbic acid | 10.00 |
| Citric acid monohydrate | 5.000 |
| Lactose monohydrate | 282.9 |
| Microcrystalline cellulose | 20.00 |
| Pregelatinized corn starch | 40.00 |
| Magnesium stearate | 2.000 |
| Industrial methylated spirit² | — |
| Water purified² | — |
| Total simvastatin layer weight | 400.0 |

Film coating

| | |
|---|---|
| OPADRY ® II Red³ | 23.28 |
| Water, purified² | — |
| Total Final Tablet Weight | 623.4 |

¹Equivalent to 50 mg of sitagliptin free base with conversion factor of 1.285.
²Removed during processing
³OPADRY Red and OPADRY Orange-Beige are based on the similar compositions of polyvinyl alcohol, talc, macrogrol/PEG and titanium dioxide.

### Method of Manufacture:

### Preparation of the Layer Containing Sitagliptin (the Sitagliptin Granulation Blend Layer):

The specific quantities of microcrystalline cellulose, croscarmellose sodium, and dibasic calcium phosphate (anhydrous) were charged with sitagliptin phosphate monohydrate to a diffusion blender. The materials were blended via diffusion (tumble) mixing. Magnesium stearate and sodium stearyl fumarate (lubricants) were screened and added to the bin blender. The blend was lubricated via diffusion (tumble) mixing to give the lubricated sitagliptin granulation blend.

### Preparation of the Layer Containing Simvastatin (the Simvastatin Granulation Blend Layer):

Granulation of a well blended mixture of simvastatin (containing 0.01% butylated hydroxyanisole), lactose, cellulose microcrystalline, and pregelatinized starch was accomplished by the addition of a hydro-alcoholic solution composed of butylated hydroxyanisole, ascorbic acid, and citric acid. The wet milled granulation was then dried and milled. Then the specific quantities of dried, milled, unlubricated simvastatin granulation and magnesium stearate (which had been screened), were charged to a diffusion blender. The materials were lubricated via diffusion (tumble) mixing to give the lubricated simvastatin granulation blend.

### Compression and Formation of the Bilayer Tablet:

The specified quantities of feed materials (lubricated simvastatin granulation blend and lubricated sitagliptin granulation blend) were compressed into bilayer tablets with the specified markings on a rotary tablet press. During the film coating step, a film-coating suspension was prepared. The compressed tablets were loaded into a coating pan and film coated with a suitable Opadry® or Opadry® II suspension (such as OPADRY® II Red) to provide the film coated tablet.

1. A pharmaceutical composition in the form of a bilayer tablet comprising:
(a) a first layer comprising about 20 to 45% by weight of sitagliptin, or a pharmaceutically acceptable salt thereof; and
(b) a second layer comprising about 5 to 15% by weight of simvastatin, or a pharmaceutically acceptable salt thereof.

2. (canceled)

3. The pharmaceutical composition of claim 1 wherein the first layer additionally comprises excipients selected from the group consisting of: (i) about 40-80% by weight of a diluent; (ii) about 0.5-6% by weight of a disintegrant; and (iii) about 0.75-10% by weight of a lubricant.

4. (canceled)

5. The pharmaceutical composition of claim 1 wherein the second layer additionally comprises excipients selected from the group consisting of: (i) about 65-85% by weight of a diluent; (ii) about 1-10% by weight of an anti-oxidant; (iii) about 5-15% by weight of a binding agent; and (iv) about 0.1-1.5% by weight of a lubricant.

6. The pharmaceutical composition of claim 1 comprising:
(a) a first layer comprising:
(i) about 20 to 45% by weight of a sitagliptin, or a pharmaceutically acceptable salt thereof;
(ii) about 40-80% by weight of a diluent;
(iii) about 0.5-6% by weight of a disintegrant; and
(iv) about 0.75-10% by weight of a lubricant; and
(b) a second layer comprising:
(i) about 5 to 15% by weight of simvastatin, or a pharmaceutically acceptable salt thereof;
(ii) about 65 to 85% by weight of a diluent;
(iii) about 1 to 10% by weight of an anti-oxidant;
(iv) about 5 to 10% by weight of a binding agent, and
(v) about 0.1 to 1.5% by weight of a lubricant.

7. The pharmaceutical composition of claim 6 wherein the diluent in the first layer is selected from the group consisting of: microcrystalline cellulose, mannitol and anhydrous dibasic calcium phosphate, or a mixture thereof; the disintegrant is selected from the group consisting of: crospovidone and croscarmellose sodium, or a mixture thereof; and the lubricant is selected from the group consisting of: magnesium stearate and sodium stearyl fumarate, or a mixture thereof.

8. The pharmaceutical composition of claim 6 wherein the diluent in the first layer is a mixture of anhydrous dibasic calcium phosphate and microcrystalline cellulose; the disintegrant is croscarmellose sodium; and the lubricant is a mixture of sodium stearyl fumarate and magnesium stearate.

9. The pharmaceutical composition of claim 8 wherein the diluent in the first layer is a mixture with a ratio of about 1:4 to about 1:6 of microcrystalline cellulose to anhydrous dibasic calcium phosphate.

10. The pharmaceutical composition of claim 6 wherein the diluent in the second layer is selected from the group consisting of: microcrystalline cellulose, lactose monohydrate and mannitol, or a mixture thereof; the anti-oxidant is selected from the group consisting of butylated hydroxyanisole, citric acid, ascorbic acid, or a mixture thereof; the binding agent is pregelatinized starch; and the lubricant is selected from the group consisting of: magnesium stearate, and sodium stearyl fumarate, or a mixture thereof.

11. The pharmaceutical composition of claim 6 wherein the diluent in the second layer is a mixture of lactose monohydrate and microcrystalline cellulose; the anti-oxidant is a mixture of butylated hydroxyanisole, citric acid monohydrate, and ascorbic acid; the binding agent is pregelatinized corn starch; and the lubricant is magnesium stearate.

12. (canceled)

13. (canceled)

14. The pharmaceutical composition of claim 1 comprising:
(a) a first layer comprising:
(i) about 25 to 35% by weight of a sitagliptin, or a pharmaceutically acceptable salt thereof;
(ii) about 50-70% by weight of a diluent;
(iii) about 1-4% by weight of a disintegrant; and
(iv) about 1.5-7% by weight of a lubricant; and
(b) a second layer comprising:
(i) about 5 to 15% by weight of simvastatin, or a pharmaceutically acceptable salt thereof;
(ii) about 70 to 80% by weight of a diluent;
(iii) about 2 to 5% by weight of an anti-oxidant;
(iv) about 5 to 15% by weight of a binding agent; and
(v) about 0.1 to 1.5% by weight of a lubricant.

15. The pharmaceutical composition of claim 14 wherein the dipeptidyl peptidase-4 inhibitor in the first layer is sitagliptin, or a pharmaceutically acceptable salt thereof; the diluent in the first layer is a mixture of anhydrous dibasic calcium phosphate and microcrystalline cellulose; the disintegrant in the first layer is croscarmellose sodium; and the lubricant in the first layer is a mixture of sodium stearyl fumarate and magnesium stearate.

16. The pharmaceutical composition of claim 15 wherein the diluent in the first layer is a mixture with a ratio of about 1:4 to about 1:6 of microcrystalline cellulose to anhydrous dibasic calcium phosphate.

17. The pharmaceutical composition of claim 14 wherein the diluent in the second layer is a mixture of lactose monohydrate and microcrystalline cellulose; the anti-oxidant in the second layer is a mixture of butylated hydroxyanisole, citric acid monohydrate, and ascorbic acid; the binding agent is pregelatinized corn starch; and the lubricant is magnesium stearate.

18. (canceled)

19. (canceled)

20. The pharmaceutical composition of claim 14 wherein the sitagliptin is present in a unit dosage strength of 50 or 100 milligrams, and the simvastatin is present in a unit dosage strength of 5, 10, 20, 40 or 80 milligrams.

21. The pharmaceutical composition of claim 14 wherein the sitagliptin is present in a unit dosage strength of 100 milligrams, and the simvastatin is present in a unit dosage strength of 10, 20, 40 or 80 milligrams.

22. The pharmaceutical composition of claim 14 wherein the sitagliptin is present in a unit dosage strength of 50 milligrams, and the simvastatin is present in a unit dosage strength of 10, 20 or 40 milligrams.

23. The pharmaceutical composition of claim 1 wherein said composition is in the dosage form of a tablet.

24. A method of treating Type 2 diabetes in a human in need thereof comprising orally administering to said human a pharmaceutical composition of claim 1.

25. (canceled)

26. The pharmaceutical composition of claim 1 wherein the bilayer tablet is coated with a film-coating agent.

27. (canceled)

28. (canceled)

29. (canceled)
